(19) United States
(12) Patent Application Publication
(10) Pub. No.: US 2008/0207594 A1
(43) Pub. Date: Aug. 28, 2008

(54) Use of Gsk-3 Inhibitors for Preventing and Treating Pancreatic Autoimmune Disorders

(75) Inventors: Rainer Mussmann, Gottingen (DE); Matthias Austen, Gottingen (DE); Arndt-Rene Kelter, Alfter (DE); Friedrich Harder, Gottingen (DE); Babette Aicher, Gottingen (DE); Alexander Lomow, Gottingen (DE)

Correspondence Address: ROTHWELL, FIGG, ERNST & MANBECK, P.C., 1425 K STREET, N.W., SUITE 800, WASHINGTON, DC, 20005, US

(73) Assignee: Davelogen Aktiengesellschaft, Goettingen (DE)

(21) Appl. No.: 11/913,612

(22) PCT Filed: May 4, 2006
(86) PCT No.: PCT/EP2006/004170

§ 371 Date: Nov. 5, 2007

(30) Foreign Application Priority Data
May 4, 2005 (EP) 05009846.6
May 30, 2005 (EP) 05011599.7
Jul. 22, 2005 (EP) 05015986.2
Oct. 24, 2005 (EP) 05023168.7

(51) Int. Cl. A61K 31/55; A61P 3/10; C12N 5/08; C12Q 1/68

(52) U.S. Cl. 514/215; 435/366; 435/6

(57) ABSTRACT

This invention relates to the use of Pax4 stimulating compounds, e.g. Glycogen synthase kinase-3 (GSK-3) inhibitors, particularly in combination with immunomodulating agents, in the prevention, and/or treatment of pancreatic autoimmune disorders, e.g. type I diabetes or LADA. More particularly, this invention relates to the use of compounds selected from paullones, indirubines, substituted ureas, maleimide derivatives and pyrimidine thiones. Further, the present invention relates to a method of identifying and/or characterizing pancreatic beta-cell mitogens by using cells expressing a pancreatic gene or a gene whose function is controlled by a pancreatic gene, particularly the Pax4 gene, and which are transfected with a reporter gene.

This invention relates to the use of Pax4 stimulating compounds, e.g. Glycogen synthase kinase-3 (GSK-3) inhibitors, particularly in combination with immunomodulating agents, in the prevention, and/or treatment of pancreatic autoimmune disorders, e.g. type I diabetes or LADA. More particularly, this invention relates to the use of compounds selected from paullones, indirubines, substituted ureas, maleimide derivatives and pyrimidine thiones. Further, the present invention relates to a method of identifying and/or characterizing pancreatic beta-cell mitogens by using cells expressing a pancreatic gene or a gene whose function is controlled by a pancreatic gene, particularly the Pax4 gene, and which are transfected with a reporter gene.

Pancreatic beta-cells secrete insulin in response to elevated blood glucose levels. Insulin amongst other hormones plays a key role in the regulation of the fuel metabolism. Insulin leads to the storage of glycogen and triglycerides and to the synthesis of proteins. The entry of glucose into muscles and adipose cells is stimulated by insulin. In patients who suffer from diabetes mellitus type I or LADA (latent autoimmue diabetes in adults, Pozzilli & Di Mario, 2001, Diabetes Care. 8:1460-67) beta-cells are being destroyed due to autoimmune attack. The amount of insulin produced by the remaining pancreatic islet cells is too low, resulting in elevated blood glucose levels (hyperglycemia). In diabetes mellitus type II liver and muscle cells loose their ability to respond to normal blood insulin levels (insulin resistance). High blood glucose levels (and also high blood lipid levels) in turn lead to an impairment of beta-cell function and to an increase in beta-cell death. Interestingly the rate of beta-cell neogenesis and replication does not appear to increase in type II diabetics, thus causing a reduction in total beta-cell mass over time. Eventually the application of exogenous insulin becomes necessary in type II diabetics.

In type I diabetics, where beta-cells are being destroyed by autoimmune attack, treatments have been devised which modulate the immune system and may be able to stop or strongly reduce islet destruction (Raz et al., 2001, Lancet 358: 1749-1753; Chatenoud et al., 2003, Nat Rev Immunol. 3: 123-132; Homann et al., Immunity. 2002, 3:403-15). However, due to the relatively slow regeneration of human beta-cells such treatments can only be successful if they are combined with agents that can stimulate beta-cell regeneration.

Diabetes is a very disabling disease, because today's common anti-diabetic drugs do not control blood sugar levels well enough to completely prevent the occurrence of high and low blood sugar levels. Frequently elevated blood sugar levels are toxic and cause long-term complications like for example nephropathy, retinopathy, neuropathy and peripheral vascular disease. Extensive loss of beta cells also leads to deregulation of glucagon secretion from pancreatic alpha cells which contributes to an increased risk of dangerous hypoglycemic episodes. There is also a host of related conditions, such as obesity, hypertension, heart disease and hyperlipidemia, for which persons with diabetes are substantially at risk.

Apart from the impaired quality of life for the patients, the treatment of diabetes and its long term complications presents an enormous financial burden to our healthcare systems with rising tendency. Thus, for the treatment of diabetes mellitus type I and LADA, but also for the treatment of late stages of diabetes mellitus type II there is a strong need in the art to identify factors that induce regeneration of pancreatic insulin producing beta-cells. These factors could restore normal function of the endocrine pancreas once its function is impaired or event could prevent the development or progression of diabetes type I, LADA or late stage diabetes type II.

The technical problem underlying the present invention was to provide for means and methods for treating pancreatic autoimmune disorders, particularly autoimmune diabetes such as type I diabetes or LADA, but also late stage type II diabetes. The solution to said technical problems is achieved by providing the embodiments characterized in the claims.

We set out to identify new molecules with beta cell regenerative capabilities by screening for small molecular weight compounds that switch on the expression of the transcription factor Pax4 in cell lines of pancreatic origin. The induction of Pax4 gene expression was chosen as a read out in the primary and secondary screening assays used in the course of the invention because the overexpression of Pax4 in human and rodent beta cells promotes beta cell proliferation and survival. Pax4 also promotes beta cell formation from stem cells in vitro and possibly neogenesis in vivo in Pax-4-transgenic mice (see, for example, WO02/086107, U.S. Pat. No. 6,071,697, U.S. Pat. No. 5,948,623, EP0958357, JP3631765, EP1288311, U.S. 60/600,704 which are incorporated herein by reference). To enable high throughput screening of compound libraries, a Pax4 reporter gene assay was established as described under Example 1. The present invention is based on the finding that structural diverse compounds, which may be GSK-3 inhibitors, for example derived from the chemical families of the paullones, the indirubins, substituted ureas and maleimide derivates stimulate the transcription of the Pax4 reporter gene construct in the adenocarcinoma cell line Capan I or the transcription of endogenous Pax4 in the insulinoma INS-1E cells as wells as in rodent islets.

The present invention is based on the finding that the above compounds stimulate the transcription of Pax4 in insulinoma INS-1E cells in vitro. Further, an increased transcription of Pax4 in rat islets was observed. An increased activity of the Pax4 gene stimulates proliferation and suppresses cell death in human beta cells. Pax4 can also stimulate beta cell formation from stem cells. The activity of Pax4 may be modulated through the effects of target molecules, e.g. GSK-3, on Pax4 activity. Inhibition or down-regulation of these target molecules results in increased Pax4 activity. Activation of Pax4 has been linked to diabetic disorders. Methods are also provided for enhanced regeneration of pancreatic beta cells through the action of the above compounds, when administered in conjunction with other immunosuppressive agents. Thus, these compounds have been identified in this invention as modulators of beta-cell regeneration.

GSK-3 exists in two isoforms, alpha and beta, which seem to have largely overlapping functions. GSK-3 has key roles in regulating a diverse range of cellular functions as the enzyme is among other things component of the insulin as well as the wnt signalling pathways. GSK-3 inhibitors have been shown to be effective in normalizing blood glucose levels in animal models of type II diabetes. An impact of GSK-3 inhibition by small molecular weight compounds or by the means of genetic methods on beta cell function in particular on beta cell replication or apoptosis, however, has not been described so far. This invention establishes a link between GSK-3 and the transcription factor Pax4 whose overexpression in beta cells is sufficient to promote beta cell growth as well as survival. GSK-3 inhibitors have been shown to protect different cell types against apoptosis induced by certain compounds or other stress factors. Moreover, there are evidences that GSK-3 alpha plays a role in the production of Alzheimer's disease amyloid peptides and new agents that specifically inhibit GSK-3 alpha are considered to be valuable in the treatment of Alzheimer's disease and may be other neurodegenerative diseases. These findings have generated an enormous amount of interest in the development of new drugs inhibiting GSK-3. In recent years a number of potent and selective GSK-3 inhibitors derived from different chemical families have reported in the literature.

The present invention relates to the use of a compound which stimulates Pax4, e.g. a GSK-3 inhibitor, particularly in combination with an immunosuppressive agent for the manufacture of a medicament for the prevention and/or treatment of autoimmune pancreatic disorders, preferably for the prevention and/or treatment of autoimmune diabetes, more preferably for the prevention and/or treatment of type I diabetes or LADA, but also for type II late-stage diabetes.

In a preferred embodiment of the invention, the compound may be a paullone. Suitable paullones are e.g. described in WO 01/60374, WO 03/027275, WO 03/099821, Meijer et al. (Handbook of Experimental Pharmacology 167 (2005), 47-64; Bertrand et al.; J. Mol. Biol. 333 (2003), 393-407; Doble and Woodgett; J. Cell. Sci. 116 (2003), 1175-1186), Meijer et al., Handbook of Experimental Pharmacology 167, 2005, 47-64; Kunick et al., J Med. Chem. 2004; 47:22-3, Kunick et al., Chembiochem. 2005; 6: 541-549, Kunick et al., Bioorganic & Medicinal Chemistry Letters 2004; 14: 413-416, which are herein incorporated by reference.

Especially preferred paullones are compounds of general formula (I)

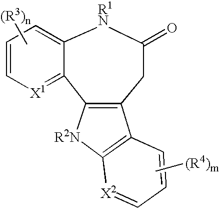

wherein X1 and X2 are independently N or CR3 and preferably X1 is N or CH and X2 is CH;
R1 and R2 are independently H, —C₁-C₆ alkyl, optionally substituted, or —CO—C₁-C₆ alkyl, optionally substituted, wherein the substituents are independently selected from one or more of halo, CN, OH, O—C₁-C₆ alkyl; COOH, COO—C₁-C₆ alkyl, —CONH₂, —CONH(C₁-C₆)alkyl, —CON(C₁-C₆ alkyl)₂, aryl, heteroaryl or combinations thereof;
each R3 and R4 is independently selected from C₁-C₆ alkyl, —C₂-C₆ alkenyl; —C₂-C₆ alkynyl; —C₃-C₁₀ cycloalkyl, —C₃-C₁₀ heterocyclyl,
aryl with 6 to 10 carbon atoms, heteroaryl with 5 to 10 ring atoms;
each optionally substituted; halo, e.g. F, Cl, Br or I; —NO₂, —CN, —OR1; —COOR1 or —NR1R2; wherein R1 and R2 are as defined above; and
wherein alkyl, alkenyl or alkynyl is optionally substituted with one or more of oxo, halo, —NO₂, —CN, —OR1, COOR1, —OCOR1, —NR1R2, NR1COR2, —NR10COR2, —NR1CONR1R2, —SR1, SOR1, —SO₂R1, —SONR1R2, SO₂NR1R2 or —NR1SO₂NR1NR2; or combinations thereof, wherein R1 and R2 are as defined above;
wherein cycloalkyl, heterocyclyl, aryl or heteroaryl is optionally substituted with one or more of C₁-C₆ alkyl, oxo, halo, —NO₂, —CN, —OR1, COOR1, —OCOR1, —NR1R2, NR1COR2, —NR10COR2, —NR1CONR1R2, —SR1, SOR1, —SO₂R1, —SONR1R2, SO₂NR1R2 or —NR1SO₂NR1NR2; or combinations thereof, wherein R1 and R2 are as defined above;
or wherein two R3 or two R4 may together form a ring;
n=0-3, preferably 0-1 and more preferably 0;
m=0-3, preferably 0, 1 or 2 and more preferably 1 or 2;
or an optical isomer or a salt thereof.

Preferably R1 and R2 are independently H, —C₁-C₅ alkyl, optionally halogenated, or CO—C₁-C₅ alkyl. More preferably R1 and R2 are H. Preferably each R3 and R4 is independently selected from C₁-C₅ alkyl, optionally halogenated, halo, —NO₂, —CN, —OR1, —COOR1, —OCOR1, —NR1NR2 and —NR1COR2. More preferably R4 is preferably selected from halo, e.g. F, Cl, Br or I; and —NO₂.

Particularly preferred examples of suitable paullones are Kenpaullone (Sigma, Cat. No. 3888), 1-Azakenpaullone (Calbiochem. Cat. No. 191500) and Alsterpaullone (Calbiochem. 1Cat. No. 26870).

In a further preferred embodiment, the compound is an indirubin. Suitable indirubins are for example described in WO 01/37819, WO 02/34717, WO 02/44148, WO 02/074742 and WO 02/100401 which are herein incorporated by reference.

Especially preferred indirubins are compounds of general formula (II)

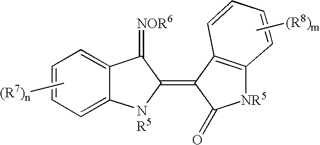

wherein R5 and R6 are independently H, —C₁-C₆ alkyl, optionally substituted, or —CO—C₁-C₆ alkyl, optionally substituted, wherein the substituents are independently selected from one or more of halo, CN, OH, O—C₁-C₆ alkyl; COOH, COOC₁-C₆ alkyl, —CONH₂, —CONH(C₁-C₆ alkyl), —CON(C₁-C₆ alkyl)₂, aryl, heteroaryl or combinations thereof;
each R7 and R8 is independently selected from C₁-C₆ alkyl, —C₂-C₆ alkenyl; C₂-C₆ alkynyl; C₃-C₁₀ cycloalkyl, —C₃-C₁₀ heterocyclyl, aryl with 6 to 10 carbon atoms, heteroaryl with 5 to 10 ring atoms, each optionally substituted; halo, e.g. F, Cl, Br or I; —NO₂, —CN, —OR1; —COOR1; or NR1R2, wherein R1 and R2 are as defined in formula (I),
wherein alkyl, alkenyl or alkynyl is optionally substituted with one or more of oxo, halo, —NO₂, —CN, —OR1, COOR1, —OCOR1, —NR1R2, NR1COR2, —NR10COR2, —NR1CONR1R2, —SR1, SOR1, —SO₂R1, —SONR1R2, SO₂NR1R2 or —NR1SO₂NR1NR2 or combinations thereof, wherein R1 and R2 are as defined in formula (I);
wherein cycloalkyl, heterocyclyl, aryl or heteroaryl is optionally substituted with one or more of C₁-C₆ alkyl, oxo, halo, —NO₂, —CN, —OR1, COOR1, —OCOR1, —NR1R2, NR1COR2, —NR1OCOR2, —NR1CONR1R2, —SR1, SOR1, —SO₂R1, —SONR1R2, SO₂NR1R2 or —NR1SO₂NR1NR2 or combinations thereof, wherein R1 and R2 are as defined in formula (I);
or two R7 or two R8 may together form a ring;
n=0-3, preferably 0-1 and more preferably 0;
m=0-3, preferably 0-1 and more preferably 1,
or an optical isomer or a salt thereof.

Preferably R5 and R6 are independently H, C₁-C₅ alkyl, optionally halogenated, or —CO—C₁-C₅ alkyl, and more preferably each R5 is H and R6 is H or COCH₃. Preferably each R7 and R8 is independently selected from C₁-C₅ alkyl, optionally halogenated; halo, e.g. F, Cl, Br or I; —NO₂, —CN, —OR5; —COOR5; —OCOR5; —NR5R6 and —NR5COR6, wherein R5 and R6 are as defined above. Most preferably R8 is selected from halo, e.g. F, Cl, Br or I.

Particularly preferred examples are GSK-3 inhibitor IX (Calbiochem, Cat. No. 361550 and FIG. 11), GSK-3 inhibitor X (Calbiochem, Cat. No. 361551) and Indirubin-3′-monoxime (Calbiochem, Cat. No. 402085).

Further, the compound may be a substituted urea, e.g. an aryl and/or heteroaryl substituted urea. Suitable substituted urea compounds are for example described in WO 03/004478, herein incorporated by reference. An especially preferred example is GSK-3b inhibitor VIII (Calbiochem, Cat. No. 361549). Especially preferred substituted ureas are compounds of general formula (III):

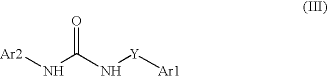

wherein Y is —[C(R9)₂]ᵣ, each R9 is independently H, F or CH₃ and r is 0-3, preferably 1,
Ar1 is an aromatic or heteroaromatic ring, preferably a 6-membered aromatic or heteroaromatic ring, more preferably a phenyl ring which is optionally substituted at least once with R7 as defined in formula (II), wherein R7 is preferably —O—C₁-C₅ alkyl optionally halogenated and/or R7 is preferably at position 4 of a phenyl ring, and

Ar2 is an aromatic or heteroaromatic ring, preferably a 5-membered heteroaromatic ring, more preferably a 1,3-thiazol ring, which is optionally substituted at least once with R7 as defined in formula (II), wherein R7 is preferably —NO₂ and/or R7 is preferably at position 5 of a thiazol ring.

Further the compound may be an ethylene diamino derivative, particularly an N,N′-diaryl or diheteroaryl substituted ethylene diamine. Especially preferred ethylene diamino derivatives are compounds of general formula (IV):

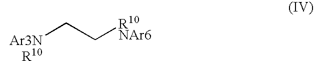

wherein each R10 is independently H, C₁-C₆ alkyl optionally substituted or —CO—C₁-C₆ alkyl optionally substituted, wherein the substituents are as defined for the substituents of R1 and R2 in formula (I),
Ar3 is an aromatic or heteroaromatic ring, preferably a 6-membered aromatic or heteroaromatic ring, more preferably a pyridine ring, e.g. a −2-pyridyl radical optionally substituted at least once, preferably once or twice, with R7 as defined in formula (II), wherein R7 is preferably selected from —NO₂, —NR5R6, CN and combinations thereof, wherein R5 and R6 are as defined in formula (II) and wherein R5 and R6 are preferably H,
Ar4 is an aromatic or heteroaromatic ring, preferably a 6-membered aromatic or heteroaromatic ring, more preferably a pyridine or a pyrimidine ring, e.g. a 2-pyridyl or a 2-pyrimidinyl radical substituted at least once, preferably once or twice with a cyclic radical selected from aryl with 6-10 carbon atoms, heteroaryl with 5-10 carbon atoms, cycloalkyl with 3-10 carbon atoms and heterocyclyl with 3-10 ring atoms, wherein the aromatic heteroaromatic ring and/or its cyclic substituents are optionally substituted at least once with R7 as defined in formula (II), wherein R7 is preferably selected from H, C₁-C₅ optionally halogenated and oxo, or an optical isomer or salt thereof.

Preferably Ar3 is a moiety of general formula (V):

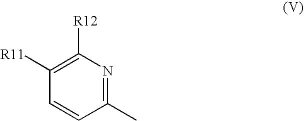

wherein R11 and R12 are independently selected from substituents defined as R7 in formula (II) and wherein R11 is preferably —NO₂ or CN and R12 is preferably —NR5R6 as defined in formula (II) and wherein R11 is preferably H.

Preferably Ar4 is a moiety of general formula (VI):

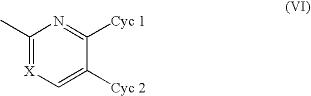

wherein X is CH or N,
Cyc 1 is aryl with 6-10 carbon atoms, preferably phenyl or heteroaryl with 5-10 carbon atoms which is at least once, preferably once or twice substituted with R5 as defined in formula (II), preferably with halo, e.g. Cl, and wherein Cyc 1 is most preferably a phenyl substituted in o- and/or in p-position as indicated above, and
Cyc 2 is heteroaryl with 5-6 ring atoms, preferably, which is optionally imidazolyl, e.g. 2-imidazolyl, at least once, preferably once, e.g. at position 5, substituted with R5 as defined in formula (II), preferably with C₁-C₅ alkyl, e.g. methyl, or
Cyc 2 is heterocyclyl with 3-6 ring atoms preferably piperazinyl, e.g. 1-piperazinyl or 1-piperazin-2-on-yl, which is optionally at least once substituted with R5 as defined in formula (II).

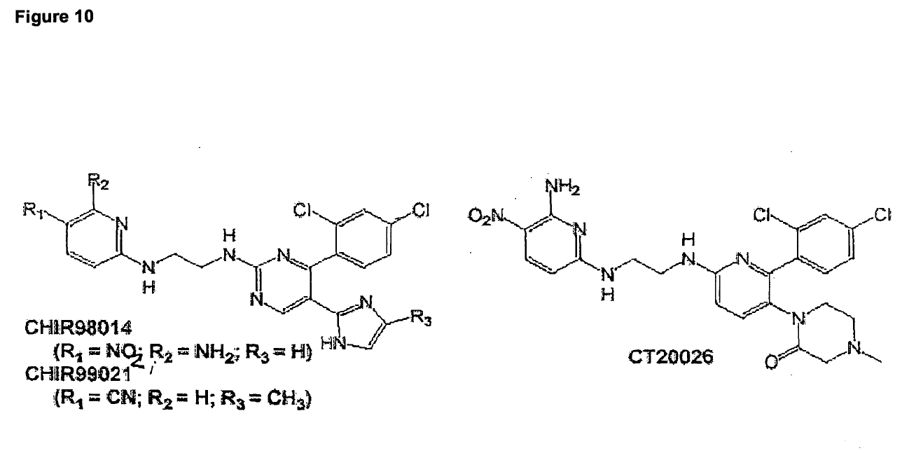

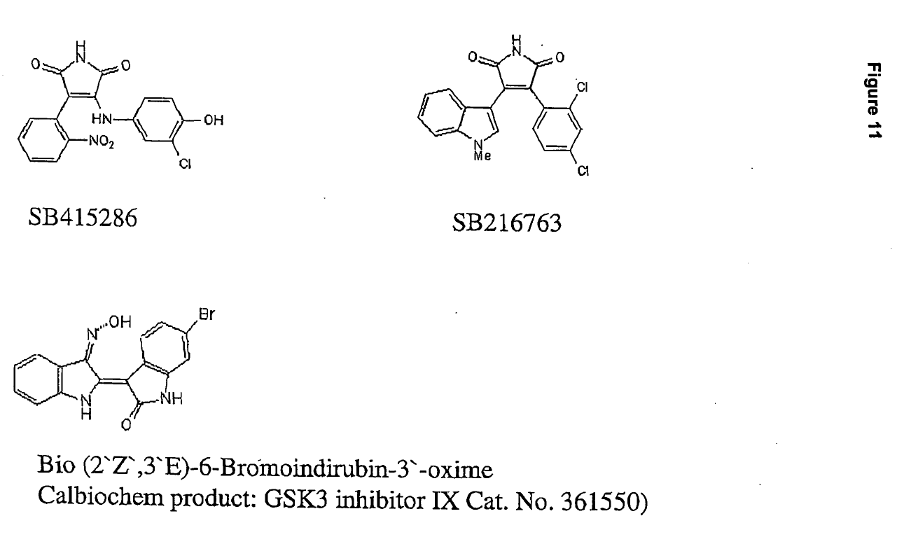

Especially preferred examples are CHIR 98014 and CHIR 99021 and CT20026 (FIG. 10).

In a still further embodiment, the compound may be a maleimide derivative. Especially preferred maleimide derivatives are compounds of general formula (VII):

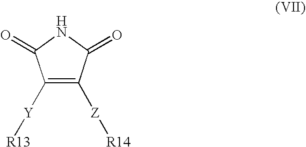

wherein R13 and R14 are independently selected from C₁-C₆ alkyl, —C₂-C₆ alkenyl, —C₂-C₆ alkynyl, —C₃-C₁₀ cycloalkyl, —C₃-C₁₀ heterocyclyl, aryl with 6 to 10 carbon atoms and heteroaryl with 5 to 10 ring atoms, each optionally substituted, wherein alkyl, alkenyl or alkynyl is optionally substituted with one or more of oxo, halo, —NO₂, —CN, —OR1, COOR1, —OCOR1, —NR1R2, NR1COR2, —NR10COR2, —NR1CONR1R2, —SR1, SOR1, —SO₂R1, —SONR1R2, SO₂NR1R2 or —NR1SO₂NR1NR2 or combinations thereof, wherein R1 and R2 are as defined in formula (I);
wherein cycloalkyl, heterocyclyl, aryl or heteroaryl is optionally substituted with one or more of C₁-C₆ alkyl, oxo, halo, —NO₂, —CN, —OR1, COOR1, —OCOR1, —NR1R2, NR1COR2, —NR10COR2, —NR1CONR1R2, —SR1, SOR1, —SO₂R1, —SONR1R2, SO₂NR1R2 or —NR1SO₂NR1NR2 or combinations thereof, wherein R1 and R2 are as defined in formula (I).

X and Y are independently selected from a chemical bond, NR1, O and S, wherein R1 is as defined in formula (I) or an optical isomer or a salt thereof.

Preferably R13 and R14 are selected from aryl or heteroaryl, e.g. phenyl or indolyl, which may be substituted e.g. with —NO₂; —OH, -halo, C₁-C₆ alkyl or combinations thereof.

Examples of suitable maleimide derivatives are disclosed in EP-B-1307447, WO 00/38675, WO 02/38561, WO 02/062387, WO 03/27275, WO 03/057202, WO 03/076398, WO 03/082859 and WO 03/103663 which are herein incorporated by reference. An especially preferred example is SB216763 (Tocris Cat. No. 1616) or SB415286 (cf. FIG. 11).

Further suitable compounds may be selected from GSK-3 inhibitors as indicated below. For example, GSK-3 inhibitors are also disclosed in “Discovery and Development of GSK3 Inhibitors for the Treatment of Type 2 Diabetes”, Allan S. Wagmann, Kirk W. Johnson and Dirksen E. Bussiere. Current Pharmaceutical Design, 2004, 10: 1105-1137, “Pharmacological inhibitors of glycogen synthase kinase 3”, Laurent Meijer, Marc Flajolet, and Paul Greengard. TRENDS in Pharmacological Sciences, 2004, 25 and references cited therein. These documents are herein incorporated by reference. In general, suitable compounds may be derived from the following chemical families:

Maleimides, e.g. SB216763 and SB415286 (SmithKline Becham) Azaindolylmaleimides, e.g. published by Kuo, G H., et al., J. Med. Chem.46, 4021-4031.

Indirubins, e.g. Indirubine-3′-monoxime
Benzazepinones (also called paullones), e.g. Azakenpaullone
Pyrroloazepines, e.g. Hymenialdisine, Aloisines
Thiazole derivates, e.g. Pyridyloxadiazole published by Naerum, L., et al.,
Bioorg. Med. Chem. Lett. 12, 1525-1528.
Flavones, e.g. Flavopiridol published by Leclerc, S., et al., JBC. 276, 251-260.
Pyrazolopyridazines published by Witherington, J., et al., Bioorg. Med. Chem. Lett. 13, 1577-1580, and 3055-3057, and 3059-3062.

### Pyrazoloquinoxalines

### Oxindoles

### Phenylaminopyrimidines

### Triazoles

### Pyrrolopyrimidines

Highly substituted purines, aminopyrimidines, and aminopyridines, e.g. CT20026 (Chiron)
5-Aryl-pyrazollopyridazines and pyridines (GlaxoSmithKline and Vertex)
Pyrazolo(3,4-b)quinoxalines
1,3,4- and 1,2,5-oxadiazoles (Novo Nordisk) and

### Thiadiazolidinones and

Pyrimidine thiones, e.g. Pyrimidin-4-yl-3,4-thiones as described in WO 2005/042525 which is herein incorporated by reference.

Moreover, GSK-3 inhibitors of diverse chemical structures have been disclosed in WO 02/50066, WO 02/22608, WO 02/22607, WO 02/22606, WO 02/22605, WO 02/22604, WO 02/22603, WO 02/22601, WO 03/37891, WO 03/37877, EP-A-1136491, EP-A-1136486, EP-A-1136485, EP-A-1136484, EP-A-1136483, EP-A-1136482, EP-A-1136099, WO 02/10141, EP-A-1256578, WO 03/11843, WO 03/24447, WO 03/04472, WO 02/65979, WO 02/50079, which are all incorporated herein by reference.

Pharmaceutically acceptable addition salts of the above compounds, e.g. the compounds (I), (II), (III), (IV) and (VII) include but are not limited to salts with physiologically acceptable cations or anions. Examples of cations are alkaline earth metals such as sodium, lithium, potassium, calcium, magnesium, aluminium salts or the like, as well as non toxic ammonium quarternary ammonium, and amine cations, including but not limited to ammonium, tetramethylammonium, tetraethylammonium, methylamine, dimethylamine, trimethylamine, triethylamine, ethylamine and the like. Other representative amines useful for the formation of base addition salts include benzazethine, dicyclohexyl amine, hydrabine, N-methyl-D-glucamine, N-methyl-D-glucamide, t-butyl amine, diethylamine, ethylene diamine, ethanolamine, diethanolamine, piperazine and the like and salts with amino acids such as arginine, lysine or the like. Examples of anions are inorganic anions such as chloride, sulphate, hydrogen sulphate, phosphate, hydrogen phosphate etc. and organic anions, e.g. carboxylate, sulphate or sulphonate anions such as acetate, lactate, tartrate, tosylate, mesylate etc.

The present invention comprises all tautomeric forms. Furthermore, the present invention also comprises all stereoisomers of the compounds according to the invention, including its enantiomers and diastereomers. Individual stereoisomers of the compounds according to the invention can be substantially present pure of other isomers, in admixture thereof or as racemates or as selected stereoisomers.

The invention also relates to metabolites and prodrugs. As used herein the term “metabolite” refers to (i) a product of metabolism, including intermediate and products, (ii) any substance in metabolism (either as a product of metabolism or as necessary for metabolism), or (iii) any substance produced or used during metabolism. In particular it refers to the end product that remains after metabolism. As used herein the term “prodrug” refers to (i) an inactive form of a drug that exerts its effects after metabolic processes within the body converts it to a usable or active form, or (ii) a substance that gives rise to a pharmacologically active metabolite, although not itself active (i.e. an inactive precursor).

As used herein the term “C₃-C₁₀ cycloalkyl” refers to mono- or polycyclic saturated or unsaturated carbocyclic alkyl groups such as cyclopropyl, cyclobutyl, cyclopentyl, cyclohexyl, cycloheptyl, cyclobutenyl, cyclopentenyl, cyclopentadienyl, cyclohexenyl, cyclohexadienyl, cycloheptenyl, cycloheptadienyl and cycloheptatrienyl and the like.

The terms “alkyl” and “alkoxy” are used herein or in combination with other terms refer to a C₁-C₆, preferably C₁-C₅ straight or branched alkyl/alkoxy group such as methyl, ethyl, propyol (iso-, n-), butyl (iso-, n-, tert-), pentyl, hexyl, methoxy, ethoxy, propoxy (iso-, n-), butoxy (iso-, n-, tert-), pentoxy, hexoxy.

The term “halogen” refers to a halogen atom selected from fluorine, chlorine, bromine, iodine, preferably fluorine and chlorine, more preferably fluorine.

The term “aryl” refers to mono- and polycyclic aromatic groups having 6 to 10 backbone carbon atoms, optionally fused to a carbocyclic group, such as phenyl, 1-naphthyl, indenyl, indanyl, azulenyl, fluorenyl, 1,2,3,4-tetrahydronaphthyl, etc.

The term “heterocyclyl” refers to mono- or polycyclic saturated or unsaturated heterocyclyl groups with 1 to 4 hetero atoms selected from N, S and O, with the remainder of the ring atoms being carbon atoms and having preferably a total number of ring atoms of 3 to 10, such as morpholino, piperazinyl, piperadinyl, pyridyl, pyrimidinyl, thiazolyl, indolyl, imidazolyl, oxadiarolyl, tetrazolyl, pyrazinyl, triazolyl, thiophenyl, furanyl, indazolyl, pyrazolopyrimidinyl, quinazolyl, etc.

The term “heteroaryl” refers to mono- or bicyclic aromatic groups with 1 to 4 hetero atoms selected from N, S and O, with the remainder of the ring atoms being carbon atoms and having preferably a total number of ring atoms of 5 to 10. Examples without limitation of heteroaryl groups are such as benzofuranyl, furyl, thienyl, benzothienyl, thiazol, imidazolyl, oxazolyl, oxadiazolyl, thiadiazolyl, benzothiazolyl, triazolyl, tetrazolyl, isoxazolyl, isothiazolyl, pyrrolyl, pyranyl, tetrahydropyranyl, pyrazolyl, pyridyl, pyrimidinyl, quinolinyl, isoquinolynyl, purinyl, carbazolyl, benzoxazolyl, benzamidazolyl, indolyl, isoindolyl, pyrazinyl, diazinyl, pyrazinyl, triazinyltriazine, tetrazinyl, tetrazolyl, benzothiophenyl, benzopyridyl, benzimidazolyl.

Thus, the compounds of the invention are considered to be mitogens and/or beta cell protective agents capable of promoting the protection, survival and/or regeneration of insulin producing cells, particularly pancreatic beta cells. In addition, the compounds also may suppress apoptotic events in beta cells thereby preventing beta cell loss. In addition, by inducing Pax4 these compounds may support beta cell neogenesis from stem or progenitor cells in vitro and in vivo.

The compound may be administered alone or in combination with other medicaments, e.g. known beta cell mitogens and/or beta cell protective agents such as GLP-1, prolactin or NGF. Further, administration may be combined with activin, e.g. activin A, activin B and/or activin AB, administration.

The compounds preserve beta cell mass and/or leads to a net increase in beta cell mass. Therefore, the compounds may be used for the prevention, amelioration and/or treatment of pancreatic autoimmune disorders, that are associated with beta cell loss.

Treatment in a medical setting could mean the direct application to patients for instance by injection. In the context of islet transplantation the agent may be used to promote survival and growth as well as differentiation of donor duct cells and islets in culture prior to or after their transfer into recipients. Another use is in stem cell differentiation protocols aiming to the production of beta cell-like cells in culture. The agent can act as a maturation factor promoting the differentiation of stem cells towards the pancreatic lineage or promoting the growth of differentiated cells.

Thus, the present invention provides methods for treating patients suffering from a pancreatic autoimmune disease caused by, associated with, and/or accompanied by functionally impaired and/or reduced numbers of pancreatic islet cells, particularly insulin producing beta-cells, by administering a therapeutically effective amount of compositions as indicated above. Functional impairment or loss of pancreatic islet cells may be due to e.g. autoimmune attack such as in diabetes type I or LADA, and/or due to cell degeneration such as in progressed diabetes type II. The methods of the present invention may also be used to treat patients at risk to develop degeneration of insulin producing beta-cells to prevent the start or progress of such process.

Numerous additional aspects and advantages of the invention will become apparent to those skilled in the art upon consideration of the following detailed description of the invention which describes presently preferred embodiments thereof.

In connection with the present invention, the term “progenitor cells” relates to undifferentiated cells capable of being differentiated into insulin producing cells. The term particularly includes stem cells, i.e. undifferentiated or immature embryonic, adult, or somatic cells that can give rise to various specialized cell types. The term “stem cells” can include embryonic stem cells (ES) and primordial germ (EG) cells of mammalian, e.g. human or animal origin. Isolation and culture of such cells is well known to those skilled in the art (see, for example, Thomson et al., (1998) Science 282: 1145-1147; Shamblott et al., (1998) Proc. Natl. Acad. Sci. USA 95: 13726-13731; U.S. Pat. No. 6,090,622; U.S. Pat. No. 5,914,268; WO 00/27995; Notarianni et al., (1990) J. Reprod. Fert. 41: 51-56; Vassilieva et al., (2000) Exp. Cell. Res. 258: 361-373). Adult or somatic stem cells have been identified in numerous different tissues such as intestine, muscle, bone marrow, liver, and brain. WO 03/023018 describes a novel method for isolating, culturing, and differentiating intestinal stem cells for therapeutic use. In the pancreas, several indications suggest that stem cells are also present within the adult tissue (Gu and Sarvetnick, (1993) Development 118: 33-46; Bouwens, (1998) Microsc Res Tech 43: 332-336; Bonner-Weir, (2000) J. Mol. Endocr. 24: 297-302).

Embryonic stem cells can be isolated from the inner cell mass of pre-implantation embryos (ES cells) or from the primordial germ cells found in the genital ridges of post-implanted embryos (EG cells). When grown in special culture conditions such as spinner culture or hanging drops, both ES and EG cells aggregate to form embryoid bodies (EB). EBs are composed of various cell types similar to those present during embryogenesis. When cultured in appropriate media, EB can be used to generate in vitro differentiated phenotypes, such as extraembryonic endoderm, hematopoietic cells, neurons, cardiomyocytes, skeletal muscle cells, and vascular cells. We have previously described a method that allows EB to efficiently differentiate into insulin-producing cells (as described in WO 02/086107 and by Blyszczuk et al., (2003) Proc Natl Acad Sci USA 100: 998-1003), which are incorporated herein by reference.

In the present invention the term “beta-cell regeneration” refers to an at least partial restoration of normal beta-cell function by increasing the number of functional insulin secreting beta-cells and/or by restoring normal function in functionally impaired beta-cells.

Before the present invention is described in detail, it is understood that all technical and scientific terms used herein have the same meanings as commonly understood by one of ordinary skill in the art to which this invention belongs.

The data disclosed in this invention show that the compositions of the invention are useful in diagnostic and therapeutic applications implicated, for example, but not limited to, pancreatic autoimmune disorders. Hence, diagnostic and therapeutic uses for the compositions of the invention of the invention are, for example but not limited to, the following: (i) tissue regeneration in vitro and in vivo (regeneration for all these tissues and cell types composing these tissues and cell types derived from these tissues), (ii) small molecule drug target, (iii) antibody target (therapeutic, diagnostic, drug targeting/cytotoxic antibody), (iv) diagnostic and/or prognostic marker, (v) protein therapy, (vi) gene therapy (gene delivery/gene ablation), and (vii) research tools.

According to this invention the composition may be administered
i) as a pharmaceutical composition e.g. enterally, parenterally or topically, preferably directly to the pancreas and/or
ii) via implantation of treated cells.

Compositions as indicated above, preferably refer to compositions comprising an active compound, e.g. a GSK-3 inhibitor optionally in combination with another medicament.

The compounds may be administered alone or in combination with another medicament useful to prevent or treat pancreatic disorders or metabolic syndrome, particularly beta-cell degeneration, for example hormones, growth factors or antioxidants such as GLP-1 and stabilized forms of GLP-1, GLP-1 analogues, DPP-IV inhibitors, nicotinamide, vitamin C, INGAP peptide, TGF-alpha, gastrin, prolactin, members of the EGF-family, or immune modulating agents such as anti-CD3 antibodies, DiaPep277 or anti-inflammatory agents such as Cox2 inhibitors, acetyl-salicylic acid, or acetaminophen. The compositions may be administered in combination with the beta cell regenerating proteins, nucleic acids and effectors/modulators thereof described in PCT/EP2004/007917, e.g. pleiotrophin and agonists thereof, or in PCT/EP2004/013175, PCT/EP2004/013535, PCT/EP 2005/000545, PCT/EP 2005/0017111 and EP 04018751.0, which are herein incorporated by reference.

More particularly, the compositions may be administered together with beta cell mitogens and/or beta cell protective agents such as GLP-1 or derivatives thereof such as GLP-1 or derivatives thereof, e.g. GLP-1 (7-36 amide), exendin-4, prolactin or neurotrophins such as NGF.

The compositions are preferably administered together with pharmaceutical agents which have an immunosuppressive activity, e.g. antibodies, polypeptides and/or peptidic or non-peptidic low molecular weight substances.

Preferred examples of immunosuppressive agents are listed in the following Table 1.

TABLE 1
Exemplary agents for immune suppression

| Names | Mechanisms |
|---|---|
| 2-amino-1,3-propanediol derivatives | Used for preventing or treating chronic |
| | rejection in a patient receiving an organ or |
| | tissue allo- or xenotransplant |
| 2-amino-2[2-(4-octylphenyl)ethyl]propane- | Immunosuppression, from accelerated |
| 1,3-diol hydrochloride | lymphocyte homing |
| 4-thiophenoxy-n-(3,4,5-trialkoxyphenyl) | Lck inhibitors |
| pyrimidine-2-amines | |
| 40-O-(2-hydroxyethyl)-rapamycin, SDZ- | Sirolimus (rapamycin) derivative, used for |
| RAD, Everolimus | acute kidney rejection; reduces rejection and |
| | graft vasculopathy following heart |
| | transplantation by inhibiting cell proliferation |
| 6-(3-dimethyl-aminopropionyl) forskolin | Immunosuppressing action useful also for |
| | treating autoimmune disease |
| 6-mercaptopurine (6-MP) | Used to treat Crohn's disease, inflammatory |
| | bowel disease and for organ transplant |
| | therapy |
| A-420983 | Lck-inhibitor |
| ABX-CBL (CBL-1) | Mouse monoclonal AB targeted against |
| | human T-cell, B-cells, NK-cells and |
| | monocytes, for treatment of steroid-resistant |
| | graft-vs-host diseases, potential use in |
| | treatment of inflammatory and autoimmune |
| | disorders |
| Alefacept (human LFA-3 IgG1 fusion | Knocks out causative memory T- |
| protein) | lymphocytes; used to treat psoriasis, a T-cell |
| | mediated inflammatory disorder |
| Antisense ICAM-1 inhibitor (ISIS 2302), | Mouse monoclonal AB blocks white blood |
| Enlimomab, BIRR1, Alicaforsen | cell adhesion to T-cell surface molecule |
| | (ICAM-1r); treatment of kidney transplant |
| | rejection |
| Antithymocyte immunoglobulin (ATGAM) | Anti-human thymocyte, immunoglobulin; |
| | used in reversal of acute kidney transplant |
| | rejection and will likely be used off-label for |
| | transplant induction therapy |
| Azathioprine | Treatment of rheumatoid arthritis and |
| | prevention of kidney transplant rejection, and |
| | other autoimmune or inflammatory disorders |
| | such as inflammatory bowel disease |
| Baohuoside-1 | Flavonoid; inhibits lymphocyte activation; Ma |
| | et al., Transplantation 78: 831-838, (2004) |
| basiliximab | Monoclonal AB that binds to receptor sites on |
| | T-cells, preventing activation by transplanted |
| | tissue (renal transplant) |
| BMS-279700 | Lck-inhibitor |
| BTI-322 | Mouse derived monoclonal AB targeted to |
| | CD2 receptor; used for prevention of first-time |
| | kidney rejection, and treatment of resistant |
| | rejection |
| Cladribine | Antimetabolite and immunosuppressive |
| | agent that is relatively selective for |
| | lymphocytes; used to treat lymphoid |
| | malignancies, e.g. hairycell leukemia |
| CP-690550 | JAK-3 inhibitor |
| Cyclophosphamide (CTX) | Immunosuppressant for treatment of arthritis |
| | and other auto-immune disorders and |
| | cancers |
| Cyclosporine (cyclosporin A, cyclosporin) | 11 amino acid cyclic peptide; blocks helper |
| | T-cell, immunosuppressant used in organ |
| | transplant therapy and other immune |
| | diseases |
| Daclizumab, HAT (Humanized Anti-Tac), | Monoclonal AB inhibits binding of IL-2 to IL-2 |
| SMART anti-Tac, anti-CD25, and | receptor by binding to IL-2 receptor; |
| humanized anti-IL2-receptor | suppresses T-cell activity against allografts |
| | (renal transplant) |
| Dexamethasone (Decadron, Dexone, | An adrenocorticoid, effective |
| Dexasone) | immunosuppressant in various disorders |
| DIAPEP-277 | Immunomodulatory properties |
| DiaMyd peptide | GAD-derived immunomodulatory peptide |
| Dipeptide Boronic Acid (DPBA) | Proteasome inhibitor; Wu et al., |
| | Transplantation 78: 360-366, (2004) |
| Docosahexaenoic acid (DHA) | Immunosuppressant that lowers the |
| | proportion of T-cells expressing CD4 or CD8, |
| | blocks antigen recognition process; Taku et |
| | al., Journal of Agricultural and Food |
| | Chemistry 48: 1047, (2000) |
| efalizumab | T-cell modulator that target T-cells through |
| | interactions with adhesion molecules on |
| | endothelial cell surface, target migration of T- |
| | cells into the skin and target activation of T- |
| | cells; used to treat Psoriasis |
| Efomycine M | Leukocyte adhesion inhibitor, Anti- |
| | inflammatory |
| FTY720 (oral myriocin derivative) | Alters lymphocyte infiltration into grafted |
| | tissues; used for prevention of organ |
| | rejection in kidney transplants |
| GAD-based vaccine/immunemodulator, | Prevention and treatment of insulin- |
| e.g. from Diamyd company | dependent diabetes |
| Glatiramer acetate (co-polymer-1) | Synthetic peptide copolymer; decoy that |
| | mimics structure of myelin so immune cells |
| | bind Copaxone instead of myelin; for multiple |
| | sclerosis |
| Glial fibrillary acidic protein (GFAP) | Possesses immunosuppressive activities in |
| | diabetic animal models; Winer et al., Nature |
| | Medicine 9: 198, (2003) |
| Gusperimus (15-deoxyspergualin) | Intravenous immunosuppressant; |
| | suppresses production of cytotoxic T-cells, |
| | neutrophils and macrophages |
| HLA-B2702 peptide | Human peptide, blocks action of NK cells and |
| | T-cell mediated toxicities, used for prevention |
| | of first kidney allograft rejection |
| hu1124(anti-CD11a) | Humanized monoclonal antibody; targets |
| | CD11a receptor on surface of T-cells to |
| | selectively inhibit immune system rejection of |
| | transplanted organs |
| hOKT31gamma (Ala-Ala) | Non Fc-binding humanized anti CD3 antibody |
| IBC-VSO1 | A synthetic, metabolically inactive form of |
| | insulin designed to prevent pancreatic beta |
| | cell destruction (vaccine) |
| IGRP-derived peptides | T-cell modulator |
| Imatinib (STI571, Glivec or Gleevec) | Lck inhibitor |
| Infliximab | Monoclonal AB, binds and inactivates human |
| | TNFalpha; used to treat Crohn's disease and |
| | rheumatoid arthritis |
| Interferon | Immunomodulatory properties |
| ISAtx247 | Used to treat autoimmune diseases such as |
| | rheumatoid arthritis and psoriasis |
| Isotretinoin | Immunosuppressant, reduces ability of T- |
| | cells to proliferate in response to immune |
| | challenge. Vergelli et al., |
| | Immunopharmacology, 31: 191, (1997) |
| L-683,742: also described as 31- | Treatment of autoimmune diseases, |
| desmethoxy-31-hydroxy-L-683,590 | infectious diseases and/or prevention o |
| | organ transplant rejections |
| Leflunomide (ARAVA) | Antiinflammatory agent |
| Medi-500 (T10B9) | Intravenous monoclonal AB that targets |
| | human T-cells; treats acute kidney rejection |
| | and graft-vs-host disease |
| Medi-507 | Intravenous humanized AB directed against |
| | CD2 T-cell; used to treat |
| | corticosteroidresistant graft-vs-host disease |
| | and prevention of kidney rejection |
| Methotrexate | Antimetabolite used to treat Crohn's disease, |
| | severe psoriasis, and adult rheumatoid |
| | arthritis (and as an anti-cancer drug) |
| Mitoxantrone | Antiproliferative effect on cellular immune |
| | system including T-cells, B-cells and |
| | macrophages; used to treat hormone- |
| | refractory prostate cancer, acute |
| | myelogenous leukemia and multiple sclerosis |
| Mycophenoiate mofetil | Inhibition of proliferation of T and B lymphocytes |
| | by blocking the synthesis of purine nucleotides; |
| | used in organ transplant therapy and inflammatory |
| | bowel disease |
| OKT4A | Mouse monoclonal AB targeted against |
| | human CD4 T-cell; used for prevention of |
| | kidney transplant rejection when used in |
| | combination with other immunosuppressant |
| | drugs |
| Oral interferon-alpha (IFN-alpha) | Early onset type 1 diabetes |
| Muromonab-CD3 | Monoclonal AB that binds to receptor sites on |
| | T-cells, preventing activation by transplanted |
| | tissue |
| Prednisolone | Corticosteroid, suppresses inflammation |
| | associated with transplant rejection |
| Psora-4 | Kv1.3-blocker |
| Rifampicin | Antibiotic; has immunomodulatory properties |
| Rituximab | CD20 antibody |
| S100beta | Possesses immunosuppressive activities in |
| | diabetic animal models |
| Sirolimus, Rapamycin | Immunosuppressant and potent inhibitor of |
| | cytokine (e.g. IL-2)-dependent T-cell |
| | proliferation (kidney transplant) |
| Tacrolimus (Prograf; FK-506) | Interferes with IL-2 TCR communication |
| Campath-1H | anti-CD52 monoclonal antibody |
| alpha-Galactosylceramide | Activation of NK-cells, immunomodulator |
| Linomide | Immunomodulator |
| Laquinimod (ABR-215062) | Linomide-derivative; immunomodulator |
| Lisofylline | antiinflammatory agent |

Preferred immunosuppressive agents are DiaPep277, anti-CD3-antibodies such as hOKT31 gamma (Ala-Ala) and GAD peptides such as DiaMyd GAD peptides.

The combination therapy may comprise coadministration of the medicaments during the treatment period and/or separate administration of single medicaments during different time intervals in the treatment period.

The compositions may be administered in patients suffering from a disease going along with reduced beta cell number and/or impaired beta-cell function, for example but not limited to one of the diseases for which a pro-proliferative effect on pancreatic beta cells and/or an anti-apoptotic/pro-survival effect on pancreatic beta cells and/or a beta cell neogenesis-promoting effect would be beneficial:
Type I diabetes: new onset, established, prevention in high-risk patients (identified e.g. via screening for multiple autoantibodies)
LADA: new onset and established
Type II diabetes: when loss of beta cell mass occurs
MODY (Maturity Onset Diabetes of the Young, all forms)
Gestational diabetes
Islet+duct cell transplantation−treatment of recipients before or after transplantation
Treatment of islets before transplantation/during pre-transplantation culture
Pancreatitis-associated beta cell loss

The compositions are also useful for in vitro and ex vivo applications for which a pro-differentiation effect on pancreatic beta cells and precursors thereof would be beneficial:
In vitro differentiation of stem cells into beta cells
In vitro transdifferentiation of duct or exocrine cells into beta cells
MODY (all forms)
Persistent Hyperinsulinemic Hypoglycemia of Infancy

More particularly, the compositions may be administered in diabetes type I, LADA or prognosed diabetes type II, but also preventively to patients at risk to develop complete beta-cell degeneration, like for example but not limited to patients suffering from diabetes type II or LADA and type I diabetes in early stages, or other types of diseases as indicated above. The compositions may also be used to prevent or ameliorate diabetes in patients at risk for type I diabetes or LADA (identified e.g. by screening for autoantibodies, genetic predisposition, impaired glucose tolerance or combinations thereof. A variety of pharmaceutical formulations and different delivery techniques are described in further detail below.

The present invention also relates to methods for differentiating progenitor cells into insulin-producing cells in vitro comprising
(a) activating one or more pancreatic genes in a progenitor, e.g. stem cell (optional step, particularly if embryonic stem cells are used)
(b) aggregating said cells to form embryoid bodies (optional step, particularly if embryonic stem cells are used)
(c) cultivating embryoid bodies or cultivating adult stem cells (e.g., duct cells, duct-associated cells, nestin-positive cells) in specific differentiation media containing a composition as indicated above under conditions wherein beta-cell differentiation is significantly enhanced, and
(d) identifying and selecting insulin-producing cells.

Activation of pancreatic genes may comprise transfection of a cell with pancreatic gene operatively linked to an expression control sequence, e.g. on a suitable transfection vector, as described in WO 03/023018, which is herein incorporated by reference. Examples of preferred pancreatic genes are Pdx1, Pax4, Pax6, neurogenin 3 (ngn3), Nkx 6.1, Nkx 6.2, Nkx 2.2, HB 9, BETA2/Neuro D, Isl 1, HNF1-alpha, HNF1-beta and HNF3 of human or animal origin. Each gene can be used individually or in combination with at least one other gene. Pax4 is especially preferred.

Further, the compositions are useful for the modulation, e.g. stimulation, of pancreatic development and/or for the regeneration of pancreatic cells or tissues, e.g. cells having exocrine functions such as acinar cells, centroacinar cells and/or ductal cells, and/or cells having endocrinous functions, particularly cells in Langerhans islets such as alpha-, beta-, delta- and/or PP-cells, more particularly beta-cells.

In a preferred embodiment, the composition, e.g. the GSK-3 inhibitor and optionally an immunosuppressive agent, can be delivered directly to progenitor, e.g. stem cells in order to stimulate the differentiation of insulin producing cells.

Further, the invention relates to a cell preparation comprising differentiated progenitor cells, e.g. stem cells exhibiting insulin production, particularly an insulin-producing cell line obtainable by the method described above. The insulin-producing cells may exhibit a stable or a transient expression of at least one pancreatic gene involved in beta-cell differentiation. The cells are preferably human cells that are derived from human stem cells. For therapeutic applications the production of autologous human cells from adult stem cells of a patient is especially preferred. However, the insulin producing cells may also be derived from non-autologous cells. If necessary, undesired immune reactions may be avoided by encapsulation, immunosuppression and/or modulation or due to non-immunogenic properties of the cells.

The insulin producing cells of the invention preferably exhibit characteristics that closely resemble naturally occurring beta-cells. Further, the cells of the invention preferably are capable of a fast response to glucose. After addition of 27.7 mM glucose, the insulin production is enhanced by a factor of at least 2, preferably by a factor of at least 3. Further, the cells of the invention are capable of normalizing blood glucose levels after transplantation into mice.

The invention further encompasses functional pancreatic cells obtainable or obtained by the method according to the invention. The cells are preferably of mammalian, e.g. human origin. Preferably, said cells are pancreatic beta-cells, e.g. mature pancreatic beta-cells or stem cells differentiated into pancreatic beta-cells. Such pancreatic beta cells preferably secrete insulin in response to glucose. Moreover, the present invention may provide functional pancreatic cells that secrete glucagon in response to hypoglycemia. A preparation comprising the cells of the invention may additionally contain cells with properties of other endocrine cell types such as delta-cells and/or PP-cells. These cells are preferably human cells.

The cell preparation of the invention is preferably a pharmaceutical composition comprising the cells together with pharmacologically acceptable carriers, diluents and/or adjuvants. The pharmaceutical composition is preferably used for the treatment or prevention of pancreatic diseases, e.g. diabetes.

According to the present invention, the functional insulin producing cells treated with compositions of the invention may be transplanted preferably intrahepatic, directly into the pancreas of an individual in need, or by other methods. Alternatively, such cells may be enclosed into implantable capsules that can be introduced into the body of an individual, at any location, more preferably in the vicinity of the pancreas, or the bladder, or the liver, or under the skin. Methods of introducing cells into individuals are well known to those of skill in the art and include, but are not limited to, injection, intravenous or parenteral administration. Single, multiple, continuous or intermittent administration can be effected. The cells can be introduced into any of several different sites, including but not limited to the pancreas, the abdominal cavity, the kidney, the liver, the celiac artery, the portal vein or the spleen. The cells may also be deposited in the pancreas of the individual.

The methodology for the membrane encapsulation of living cells is familiar to those of ordinary skill in the art, and the preparation of the encapsulated cells and their implantation in patients may be accomplished without undue experimentation. See, e.g., U.S. Pat. Nos. 4,892,538, 5,011,472, and 5,106.627, each of which is specifically incorporated herein by reference. A system for encapsulating living cells is described in PCT Application WO 91/10425 of Aebischer et al., specifically incorporated herein by reference. See also, PCT Application WO 91/10470 of Aebischer et al., Winn et al., Exper. Neurol., 1 13:322-329, 1991, Aebischer et al., Exper. Neurol., 11 1:269-275, 1991; Tresco et al., ASAIO, 38: 17-23, 1992, each of which is specifically incorporated herein by reference. Techniques for formulating a variety of other sustained- or controlled-delivery means, such as liposome carriers, bio-erodible particles or beads and depot injections, are also known to those skilled in the art.

Immunomodulating medicaments, e.g. immunosuppressive drugs, such as cyclosporin, are preferably administered to the patient in need to reduce the host reaction versus graft. Allografts using the cells obtained by the methods of the present invention are also useful because a single healthy donor could supply enough cells to regenerate at least partial pancreas function in multiple recipients.

Administration of the pharmaceutical compositions to a subject in need thereof, particularly a human patient, leads to an at least partial regeneration of pancreatic cells. Preferably, these cells are insulin producing beta-cells that will contribute to the improvement of a diabetic state. With the administration of this composition e.g. on a short term or regular basis, an increase in beta-cell mass can be achieved. This effect upon the body reverses the condition of diabetes partially or completely. As the subject's blood glucose homeostasis improves, the dosage administered may be reduced in strength. In at least some cases further administration can be discontinued entirely and the subject continues to produce a normal amount of insulin without further treatment. The subject is thereby not only treated but could be cured entirely of a diabetic condition. However, even moderate improvements in beta-cell mass can lead to a reduced requirement for exogenous insulin, improved glycemic control and a subsequent reduction in diabetic complications.

Preferably, the compositions of the invention are intended for pharmaceutical applications and may comprise with a pharmaceutically acceptable carrier, for any of the therapeutic effects discussed above. Such pharmaceutical compositions may consist of the active ingredient of the invention. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone or in combination with other agents, drugs or hormones. The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual or rectal means.

In addition to the active ingredients, these pharmaceutical compositions may contain suitable pharmaceutically-acceptable carriers comprising excipients and auxiliaries, which facilitate processing of the active compounds into preparations, which can be used pharmaceutically. Further details on techniques for formulation and administration may be found in the latest edition of Remington's Pharmaceutical Sciences (Maack Publishing Co., Easton, Pa.).

Pharmaceutical compositions suitable for use in the invention include compositions wherein the active ingredients are contained in an effective amount to achieve the intended purpose. The determination of an effective dose is well within the capability of those skilled in the art. For any compound, the therapeutically effective dose can be estimated initially either in cell culture assays, e.g., of pancreatic cells or in animal models, usually mice, rabbits, dogs or pigs. The animal model may also be used to determine the appropriate concentration range and route of administration. Such information can then be used to determine useful doses and routes for administration in humans. A therapeutically effective dose refers to that amount of active ingredient, which is sufficient for treating a specific condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week or once every two weeks depending on half-life and clearance rate of the particular formulation. Normal dosage amounts may vary from 0.1 to 100,000 μg, up to a total dose of about 1 g, depending upon the route of administration. Guidance as to particular dosages and methods of delivery is provided in the literature and generally available to practitioners in the art.

Furthermore, the present invention relates to a method of identifying and/or characterizing beta-cell mitogens by using a cell transfected with a reporter gene construct comprising an expression control sequence, e.g. a pancreatic expression control sequence such as the Pax4 expression control sequence operatively linked to a reporter gene.

In particular, the invention relates to a method of identifying and/or characterizing a pancreatic beta-cell mitogen comprising the steps:
(i) providing a cell which is transfected with a reporter gene construct comprising a reporter gene which is operatively linked to an expression control sequence of a pancreatic gene or a gene whose function is controlled by a pancreatic gene, preferably the Pax4 gene,
(ii) contacting said cell with a compound and
(iii) determining reporter gene expression in said cell as a response to the presence of the compound.

The cell capable of regulating the expression level of a pancreatic gene or a gene whose function is controlled by a pancreatic gene, particularly the Pax4 gene, is preferably of pancreatic origin, derived from a beta cell or a precursor thereof, an insulinoma or insulinoma derived cell. The cell is preferably of mammalian origin such as a rat cell, e.g. INS-1 (Asfari et al. (1992), Endocrinology 130:167-178), mouse cell, e.g. a NIT-1 cell (Hamaguchi et al. (1991), Diabetes 40: 842-849), or a human cell, in which the expression of the endogenous Pax4 is inducible, e.g. by activins or betacellulin (Ueda (2000), FEBS Lett. 480:101-105), and which have been transfected with a suitable reporter construct. Especially preferred are transgenic cell lines containing said reporter construct which exhibits increased Pax4 reporter gene activity after treatment with activators like activin, betacellulin or kinase inhibitors, such as INS-1-cl.-1.5 or INS-1-cl.-9.

The reporter gene construct comprises an expression control sequence, and optionally part of the gene locus, of a pancreatic gene or a gene whose function is controlled by a pancreatic gene, preferably the Pax4 promoter and the Pax4 gene locus. Further non-limiting examples of pancreatic genes are Pdx1, Pax-4, Pax-6, neurogenin 3 (ngn3), Nkx×6.1, Nkx×6.2, Nkx×2.2, HB9, BETA2/NeuroD, Isl1, HNF1-alpha, HNF1-beta, HNF3, HNF4 alpha, Hes1 and H1xb9 or IRS2, c-myc, a cyclin, a CDK inhibitory protein, Menin1 and CDK4 of mammalian or human origin.

The reporter gene may be any gene expressing a reporter gene product which gives a phenotypically detectable signal, e.g. a signal which can be detected by optical or enzymatic methods. Preferred examples of reporter genes are the firefly luciferase gene, the chloramphenicol transferase gene (CAT) or beta galactosidase gene (Current Protocols In Molecular Biology, Ausubel, I., Frederick, M. (1999); John Wiley & Sons, Inc.; Introduction of DNA into Mammalian Cells Overview of Genetic Reporter Systems; page 9.6.3). The reporter gene is heterologous to the expression control sequence.

Preferably, the inventive method is performed in vitro, e.g. in a cell culture system. The method may also be performed in vivo in a non-human transgenic animal.

The reporter gene construct may be inserted into an appropriate vector, i.e. a vector which allows propagation and expression in an insulin producing cell. Methods which are well known to those skilled in the art may be used to construct suitable vectors containing sequences encoding the proteins and the appropriate transcriptional and translational control elements. Such techniques are described in Sambrook, J. et al. (1989) Molecular Cloning, A Laboratory Manual, Cold Spring Harbor Press, Plainview, N.Y. And Ausubel, F. M. et al. (1989) Current Protocols in Molecular Biology, John Wiley & Sons, New York, N.Y.

Appropriate mammalian expression vectors for efficient expression, selection, and analysis of recombinant proteins are well known in the art (Sambrook, J. et al. (1989) Molecular Cloning, A Laboratory Manual, Cold Spring Harbor Press, Plainview, N.Y.), such as vector systems based on viruses such as retroviruses or adenoviruses. Another type of vector that is suitable for transfection is known in the art as plasmid expression or cloning vectors, e.g. Bluescript vectors (Stratagene) or plasmid pCMV-SPORT (Invitrogen), is commercially available from a number of different suppliers.

The reporter gene construct can be either transiently or stably inserted into the cell by any suitable method. Transfection methods are well known in the art (see, for example, Ausubel et al., 1991, Current Protocols in Molecular Biology, Wiley and Sons, New York). Preferably, the cell is stably transfected with the reporter gene construct.

The transfected cells according to the present invention are contacted with a test compound under conditions wherein the effect of the test compound on the reporter gene product expression can be determined. Preferably at least 10⁴ cells, more preferably at least 2×10⁴ cells are used for each test. The test compound may be dissolved in a buffer, medium or solvent which is physiologically acceptable for the cells and then incubated with the cells for a suitable time period, e.g. of about 1 hour to about 80 hours, preferably from about 3 hours to about 60 hours, most preferably about 6 to about 48 hours.

After the contacting step, the reporter gene expression is determined. The determination comprises a qualitative and/or quantitative detection of a gene product which is formed upon expression of the reporter gene. If an increased reporter gene expression versus a control, e.g. reporter gene expression in the absence of a test compound, is found, the test compound can act as a pancreatic beta-cell mitogen. The reporter gene expression may be determined by any suitable optical or enzymatic method well known in the art.

The present invention is suitable for automatised cell-based (high through-put and ultra high through-put) screening assays which are well known in the art. With the present method, a plurality of compounds can be screened in parallel within a very short time. Surprisingly it was found that the method disclosed in the present invention is easy, accurate and quick.

Insulinoma derived cells are valuable cellular systems for identifying and/or characterizing pancreatic beta-cell mitogens. Molecules capable of inducing the expression of a pancreatic gene or a gene whose function is controlled by a pancreatic gene, such as Pax4 in pancreatic beta-cells or precursors thereof are putative beta cell mitogens. Cells, e.g. insulinoma cells that are transfected with an inducible Pax4 gene are among the best cellular systems to identify and/or characterize beta cell mitogens, in particular when the Pax4 expression can be induced by physiological stimuli, such as with activins, beta-cellulin, prolactin, glucose or GLP-1, because these cells are expected to comprise Pax4 regulative signalling pathways alike real beta-cells.

Thus, molecules activating pancreatic gene expression or the expression of a gene whose function is controlled by a pancreatic gene such as Pax4 expression in said cells, e.g. insulinoma cells, are also expected to be effective in primary beta-cells and in vivo. The invention described herein facilitates the identification and/or characterization of novel pancreatic beta-cell mitogens, e.g. it can be used to screen large chemical or compound libraries for beta cell mitogens, particularly Pax4 agonists.

A further aspect of the present invention relates to an insulinoma cell which is transiently or stably transfected with a reporter gene which is operatively linked to an expression control sequence and, optionally, part of the gene locus of a pancreatic gene or a gene whose function is controlled by a pancreatic gene. Preferably, the reporter gene comprises the Pax4 promoter and the Pax4 gene locus, as well as a reporter gene sequence, particularly of human origin.

An even further aspect of the present invention relates to a test system for the identification and characterization of a beta-cell mitogen comprising an insulinoma derived cell which is stably transfected with a reporter gene which is operatively linked to an expression control sequence of a pancreatic gene or a gene whose function is controlled by a pancreatic gene, preferably the Pax4 gene, and at least one positive or negative control.

In a preferred embodiment, the expression control sequence of the test system is of mammalian, preferably human origin. The reporter gene of the test system comprises a sequence coding for, amongst others, a firefly luciferase, chloramphenicol acetyltransferase or a beta galactosidase gene (Current Protocols In Molecular Biology, Ausubel, I., Frederick, M. (1999); John Wiley & Sons, Inc.; Introduction of DNA into Mammalian Cells: Overview of Genetic Reporter Systems; page 9.6.3).

In a further preferred embodiment, the control compounds are selected from, amongst others, activin A, B, AB, C or D, TGF beta, HGF, IGF, prolactin, GLP-1 or derivatives thereof, EGF, betacellulin, glucose or a small molecule kinase inhibitor such as inhibitor I, II or III, or combinations thereof.

It should be noted that all preferred embodiments discussed for one or several aspects of the invention also relate to all other aspects.

The figures illustrate the invention:

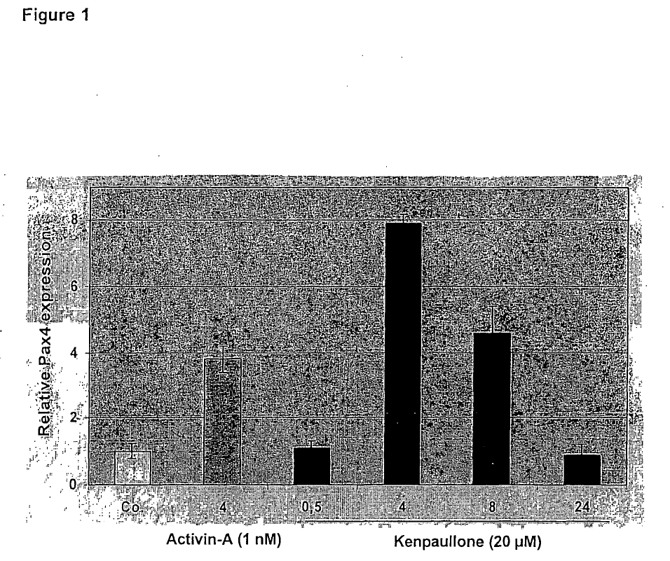

FIG. 1 illustrates a representative experiment in which the relative Pax4 levels were quantified using quantitative real time RT-PCR. As shown in FIG. 1, Kenpaullone (20 μM) transiently induces Pax4 gene transcription in INS-1E cells. Data are presented as relative levels to the basal Pax4 expression level in untreated (Co.) and in activin A (1 nM) treated INS-1E cells.

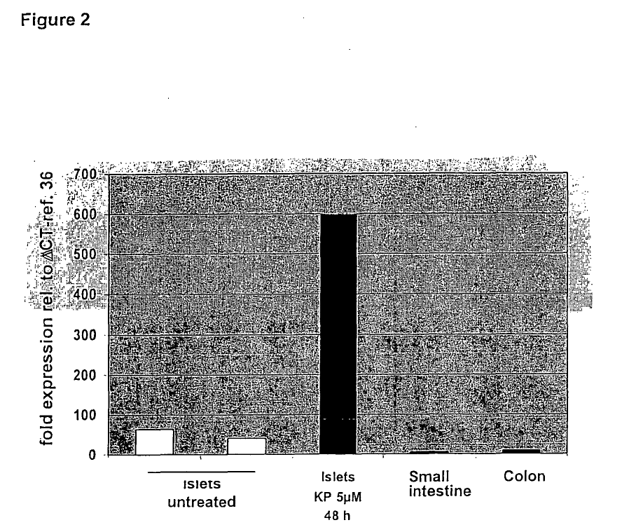

FIG. 2 illustrates relative Pax4 expression in untreated human islets and human islets, treated with 5 μM Kenpaullone (KP) for 48 h.

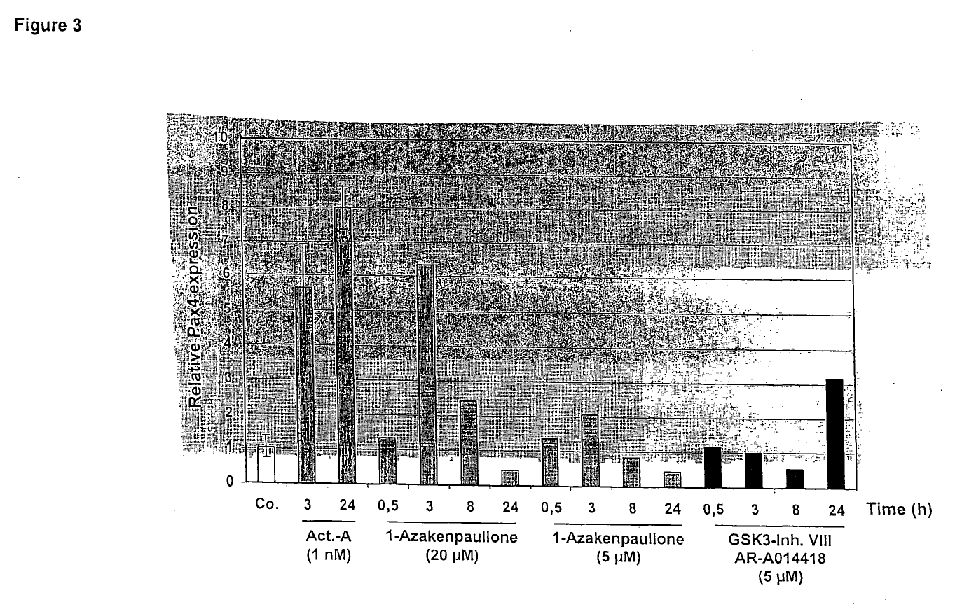

FIG. 3 shows the relative Pax4 levels in INS-1E cells treated with activin A (Act A), 1-Azakenpaullone and GSK-3 inhibitor VIII.

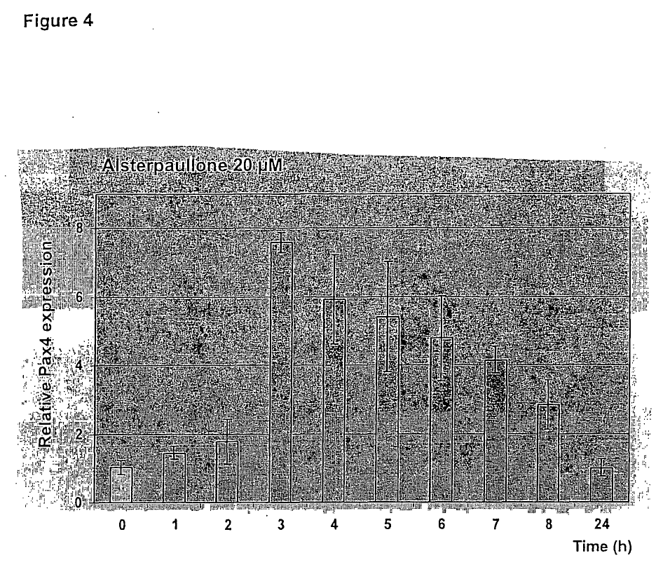

FIG. 4 illustrates the relative Pax4 expression in INS-1E cells after treatment with 20 μM Alsterpaullone for the indicated time.

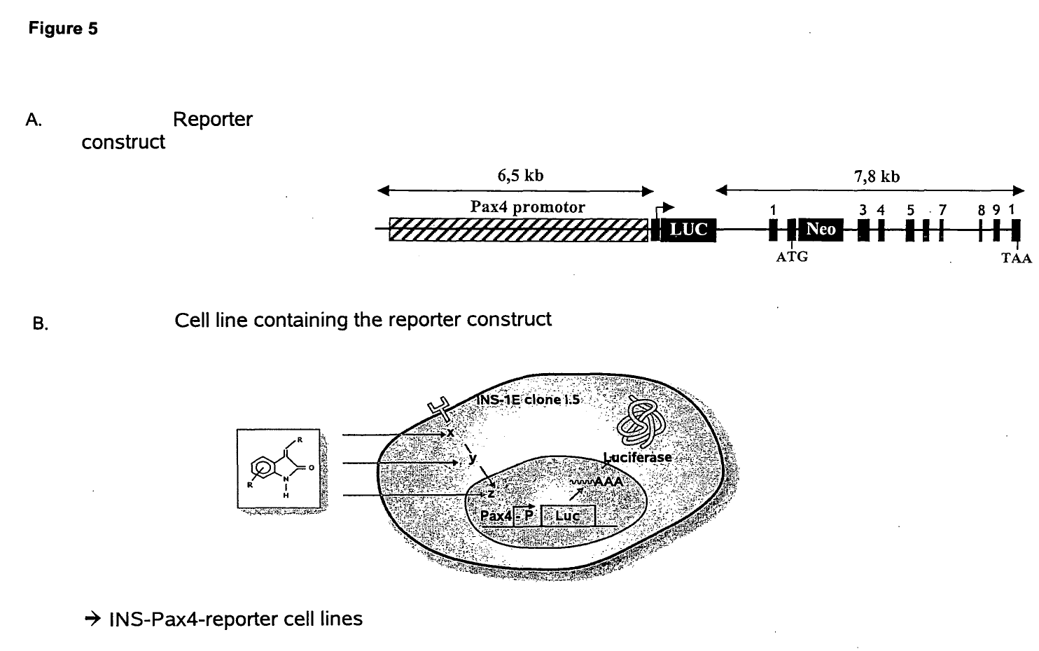

FIG. 5A illustrates the Pax4 reporter gene construct. Pax4 promotor sequence from −6500 to +1 were ligated upstream of a fire fly luciferase (LUC) reporter gene and 7.8 kb of the genomic Pax4 gene locus. The 17.7 kb cDNA construct was cloned into pBlueskript KS. The numbers 1 to 10 indicate human Pax4 exons.

FIG. 5B shows a schematic illustration of an insulinoma cell carrying a Pax-4-reporter gene construct.

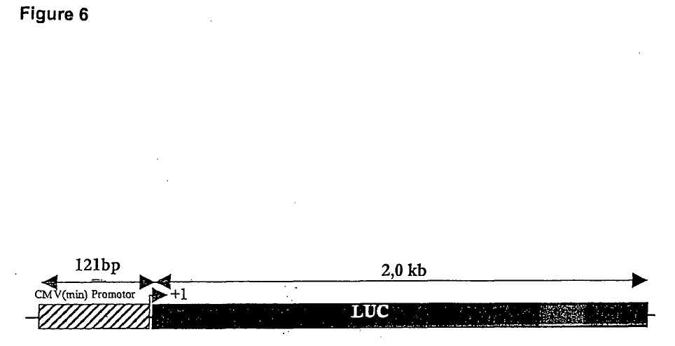

FIG. 6 shows the pCMV(min)-LUC reporter gene construct. Minimal CMV promoter sequences (121 bp) were ligated upstream of the fire fly luciferase reporter gene and cloned into pcDNA3.1 without internal CMV promotor.

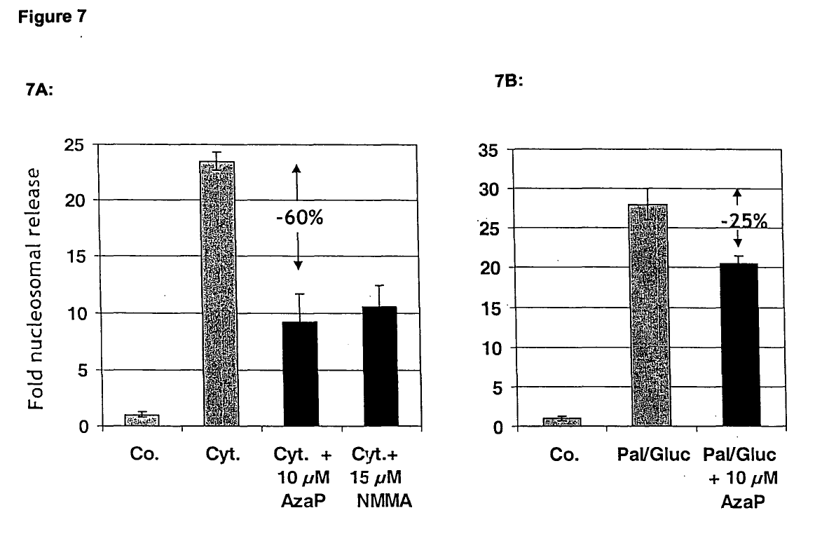

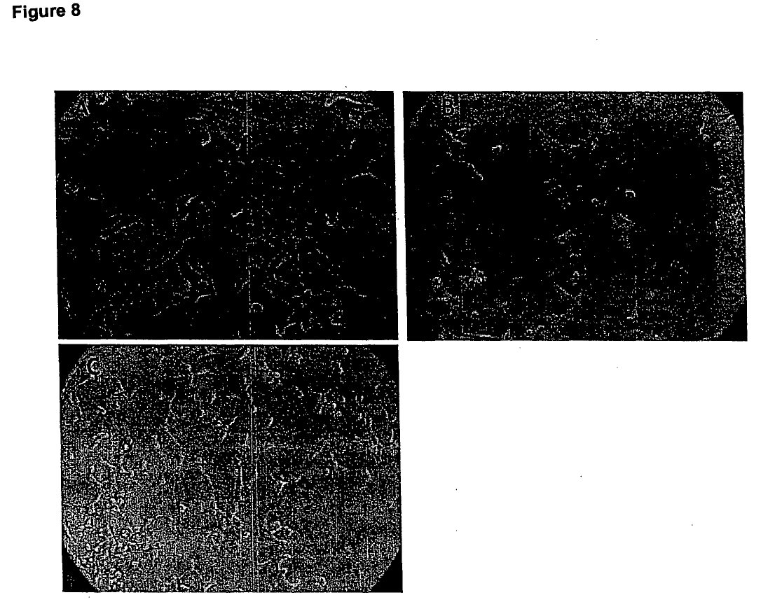

FIG. 7: Determination of cytokine or high glucose and fatty acid induced Nucleosomal-fragmentation in INS-1 cells. To induce apoptosis INS-1 cells were exposed to the cytokines IL-1 beta at 4 ng/ml and IFN-gamma at 1 U/ml (Cyt. in figure A) or glucose at 25 nM and palmitate at 0.04 nM (Pal/Gluc in figure B) for 24 hours. 1 hour before the application of these reagents indicated cell samples were treated with 10 μM 1-Azakenpaullone (AzaP) or 15 μM NG-monomethyl-L-arginine monoacetate (L-NMMA) an inhibitor of the inducible nitric oxide synthase (iNOS). Apoptosis was assessed by determination of nucleosomal accumulation in the cytosolic extracts using a cell death detection ELISA kit. Nucleosomal release is expressed relative to untreated control cells (Co.).

FIG. 8: Cell morphology of treated cells was inspected by light microscopy. Cell morphologies of untreated (A), Pal/Gluc (B) and Pal/Gluc+AzaP (C) treated cells are shown.

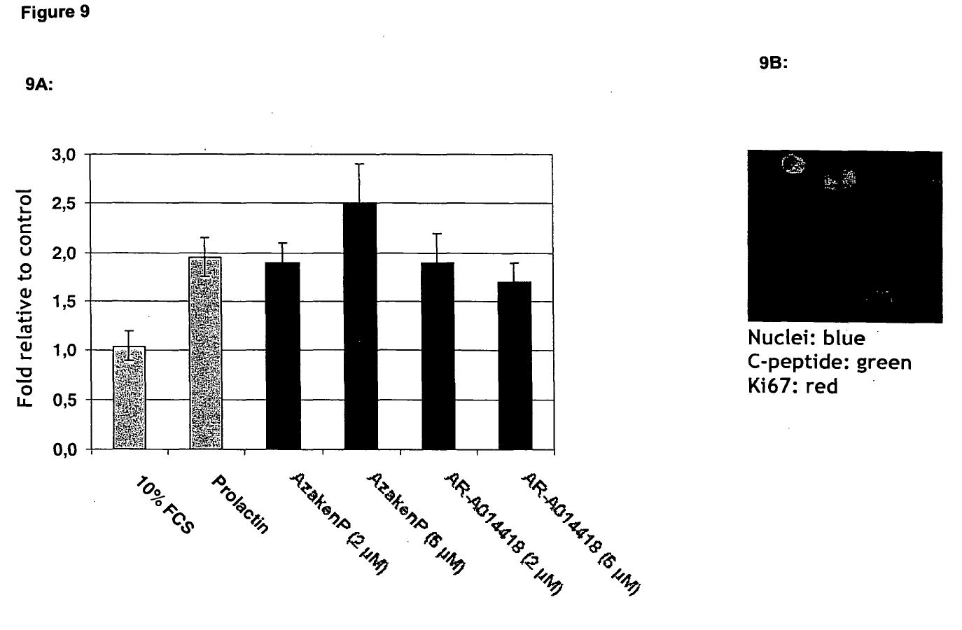

FIG. 9 shows the quantization of beta cell proliferation based on Ki-67 and C-peptide staining of replicating beta cells. About 1.5×10⁴ beta cells were inspected per experiment and the percentage of double positive cells was determined. The number of replicating beta cells is expressed relative to control cells maintained in 10% FCS (FIG. 9A). Prolactin treated cells served as a technical control. Identification of C-peptide and Ki67 double positive beta cells dispersed on adhesive slides was carried out by immunohistochemistry (FIG. 9B).

FIG. 10 shows the inhibitors CHIR98014 (R₁=NO₂; R₂=NH₂; R₃=H); CHIR99021 (R₁=CN; R₂=H; R₃=CH₃); CT20026.

FIG. 11 shows the inhibitors SB415286, SB216763, Bio (2′Z′,3′E)-6-Bromoindirubin-3′-oxime (Calbiochem product: GSK3 inhibitor IX Cat. No. 361550).

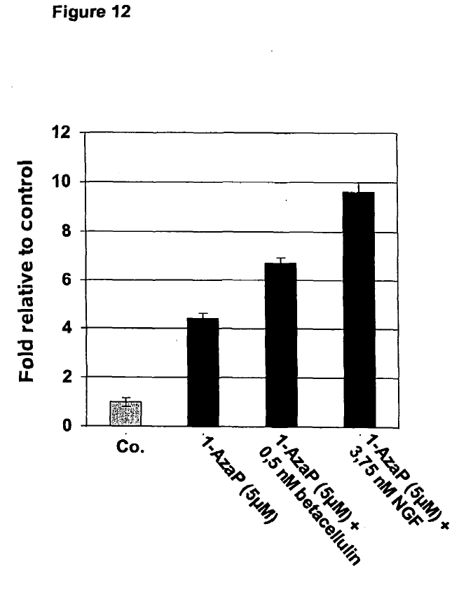

FIG. 12 illustrates synergistic effects of 1-Azakenpaullone and nerve growth factor (NGF) or betacellulin on the replication of primary rat beta cells. The results suggest that NGF and to a lesser extent betacellulin potentiate the growth promoting effects of the GSK3 inhibitor 1-Azakenpaullone. In our hands, betacellulin alone does not influence replication of primary rat beta cells (data not shown). Likewise, NGF alone is also not sufficient to trigger beta cell replication. The experimental set up used for these studies is identical to this described in FIG. 9, except that the incubation period was extended from 48 to 72 hours. The diagram shows that GSK3 inhibitors and NGF or betacellulin synergistically promote beta cell replication in rat islets.

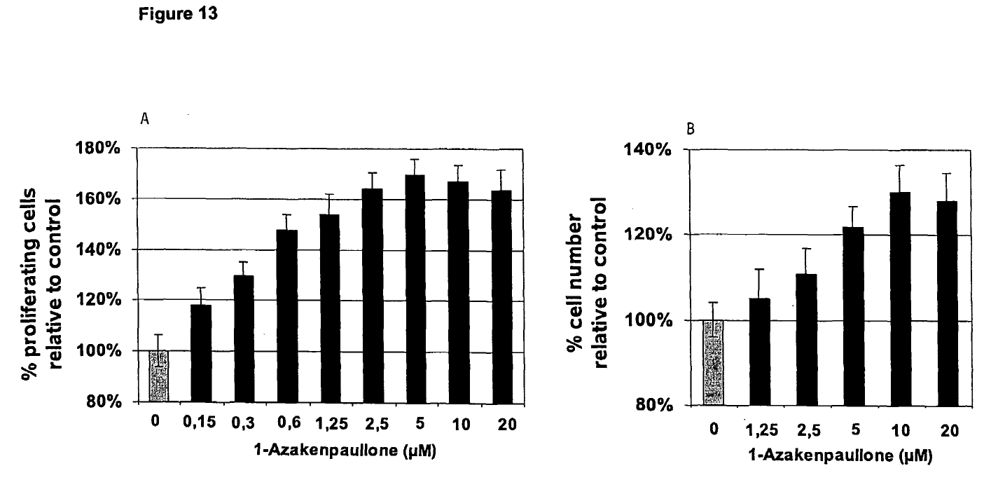

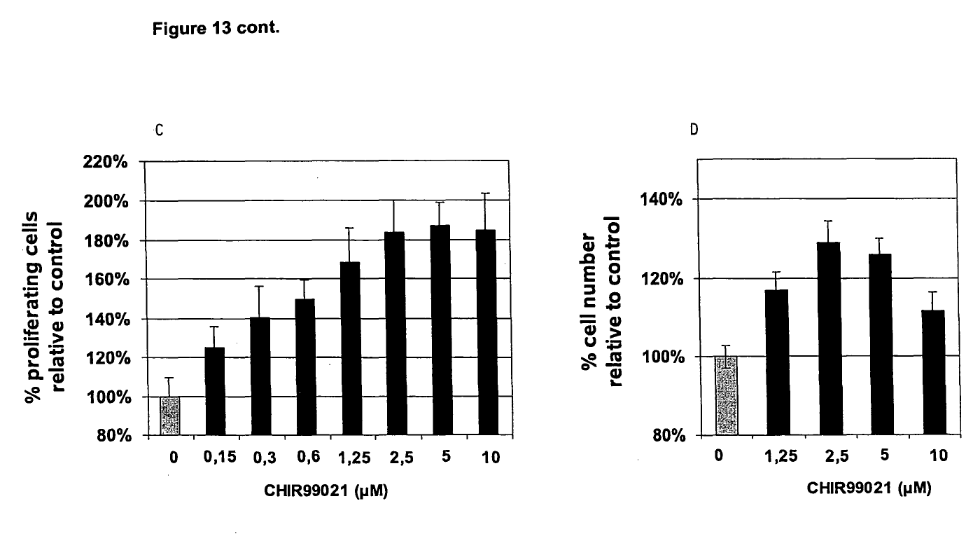

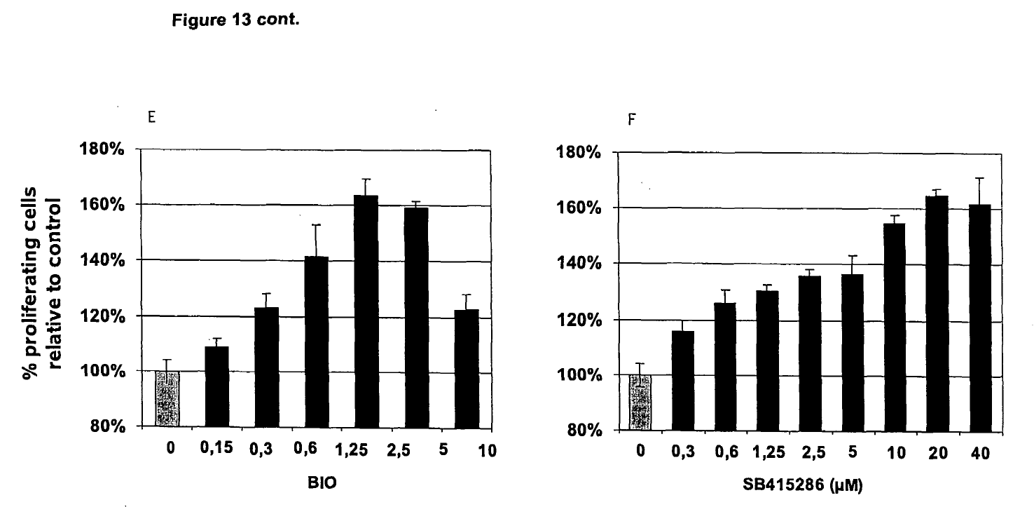

FIG. 13: GSK3 inhibitors promote replication and growth of INS-1E cells in a concentration dependent manner. INS-1 is a rat insulinoma cell line that responses to certain beta cell mitogenic factors by rising its rate of replication (Wang Q., et al.; Diabetologia 47 (2004), 478-487; Huotari M., et al., Endocrinology 139 (1998), 1494-1499). It was found that this also applies to the INS-1E subclone that was used in studies described herein. Regulation of INS-1E cell proliferation by GSK3 inhibitors was determined by incorporation of BrdU using the BrdU labeling and detection assay (Roche) as well as by cell counting using CyQuant-assay (Molecular Probes). In brief, 20.000 INS-1E cells were seeded per well of a 96 well culture dish. After 24 hours cultivation the medium containing 10% FCS serum was replaced with medium containing 1% serum before the compounds were applied. After additional 20 hours of cultivation BrdU was added to the medium for 4 hours before harvesting. The relative cell numbers were determined using the CyQuant assay after cells were grown for 4 days in the presence or absence of GSK3 inhibitors.

FIGS. 13A and 13B: 1-Azakenpaullone

A: BrdU labeling and detection assay
B: CyQuant assay

FIGS. 13C and 13D: CHIR99021 (compound illustrated in FIG. 10)

C: BrdU labeling and detection assay
D: CyQuant assay

FIG. 13E: BIO ((2′Z,3′E)-6-Bromoindirubin in 3′-oxime)

BrdU labeling and detection assay

FIG. 13D: SB415286

BrdU labeling and detection assay

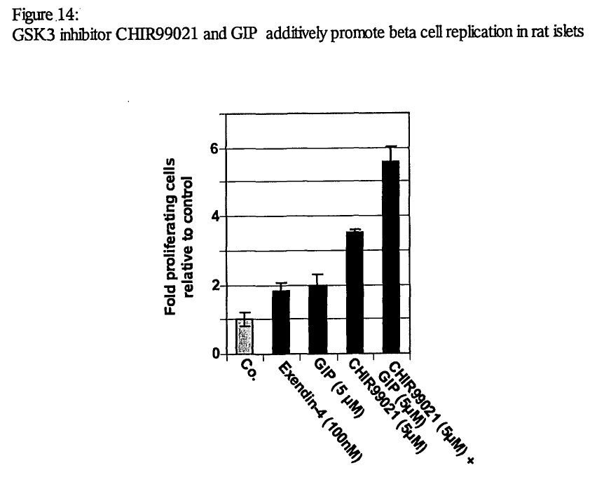

FIG. 14 illustrates the cooperative effects of the GSK3 inhibitor CHIR99021 and the incretin GIP on the replication of primary rat beta cells. CHIR99021, GIP or exendin-4 alone also trigger beta cell replication though to a lesser extend than the combination of CHIR99021 and GIP. The experimental set up used for these studies is identical to this described in FIG. 9, except that the incubation period was extended from 48 to 72 hours.

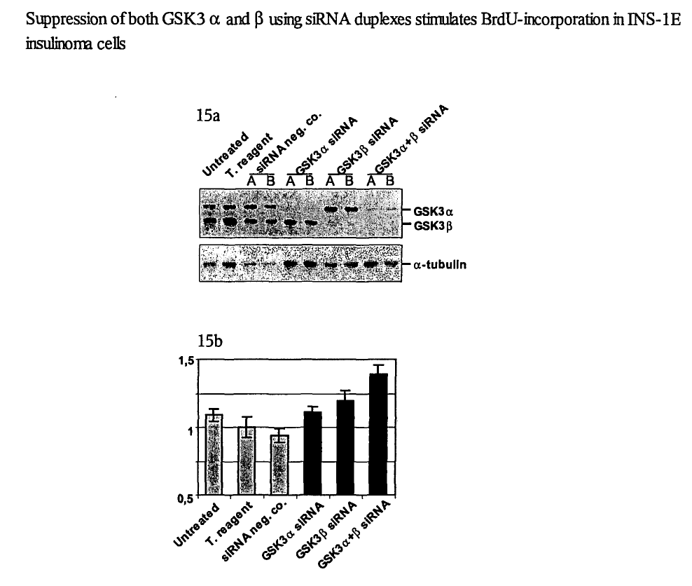

FIG. 15A shows a western blot incubated with antibodies recognizing GSK3 α and β. The expression of GSK3 α and β was suppressed in INS-1E cells using gene specific siRNA duplexes (GSK3 α or β siRNA). The expression of GSK3 isoforms was compared to this in INS-1E cells not treated with transfection reagents or siRNA (untreated) or treated with the transfection reagent alone (T. reagent) or control non-silencing siRNA duplexes provided by the supplier (siRNA neg. co.). Staining with an γ-tubulin antibody confirmed equal loading of the wells. FIG. 15B shows that only the simultaneous suppression of both GSK3 isoforms stimulates the proliferation of INS-1E cells.

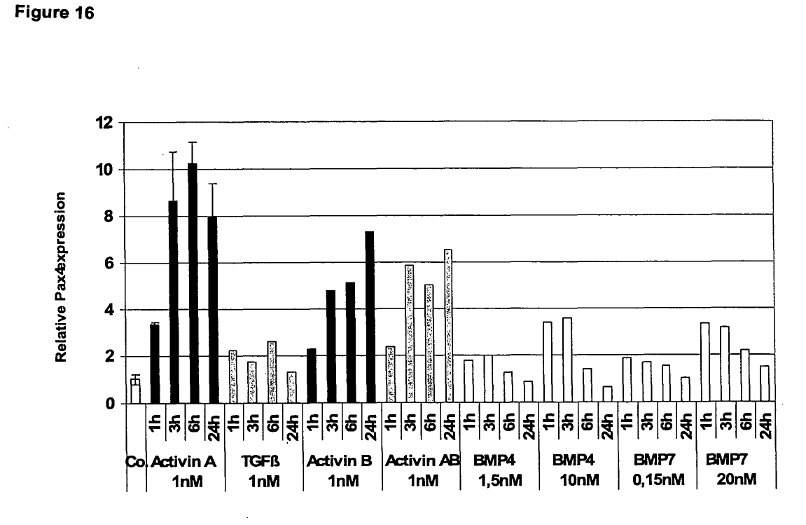

FIG. 16 shows the Pax4 RNA expression level in insulinoma INS-1E cells without treatment (Co) or after treatment with activin A, TGF-beta, activin B, activin AB, BMP-4 and BMP-7 in concentrations as indicated. Pax4 expression levels were quantitatively determined by real time RT-PCR and are indicated in relative amounts.

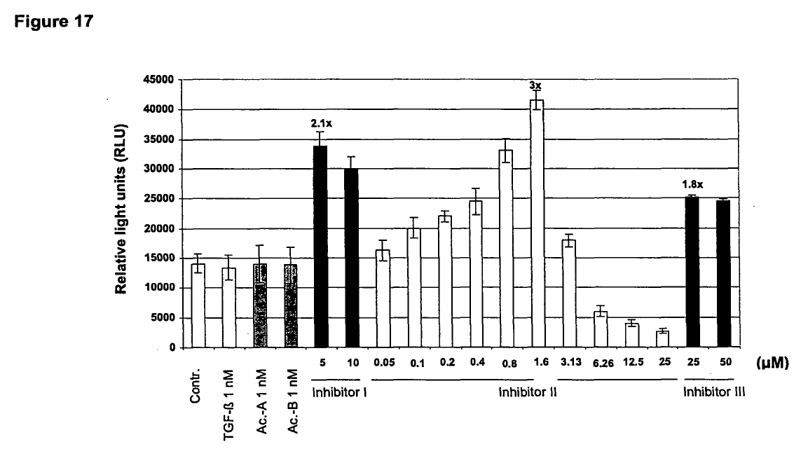

FIG. 17 shows the induction of reporter gene (luciferase) activity in the cell INS-1-cl.-1.5 by several test compounds.

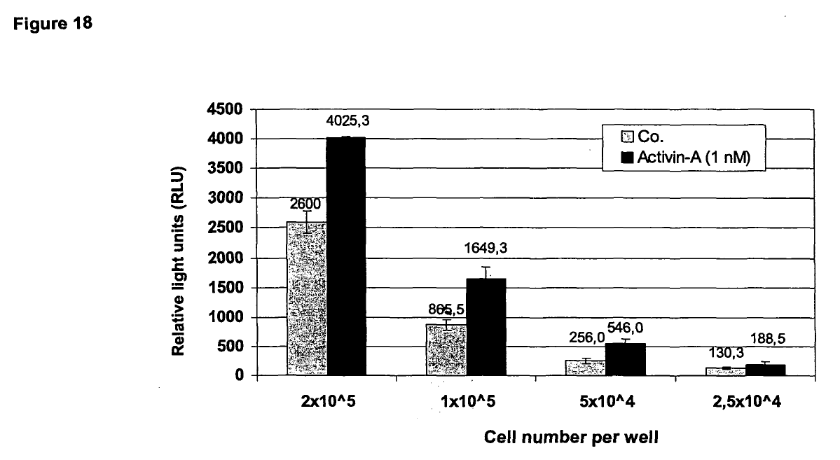

FIG. 18 shows the activity in the transgenic cell line INS-1-cl.-3.5 after treatment with activin-A. The best signal to noise ratio is achieved when 0.5×10⁵ to 1×10⁵ cells are seeded per well (0.3 cm²) of a so-called 96 well plate.

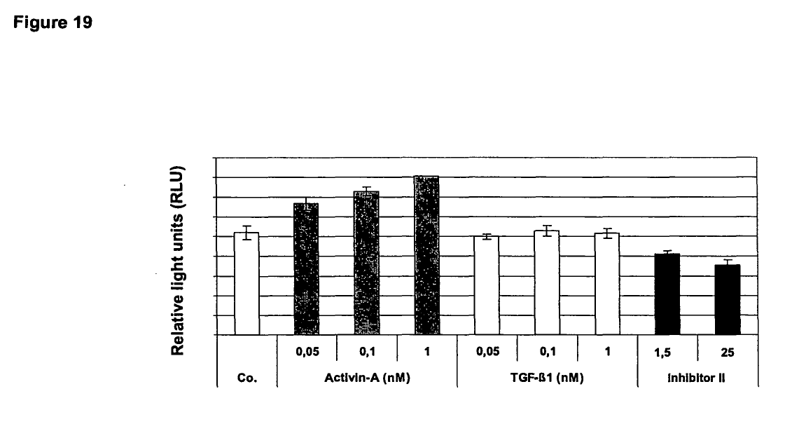

FIG. 19 shows the induction of reporter gene activity in the cell line INS-1-cl.-3.5 by treatment with activin-A, TGF-beta and kinase inhibitor 11.

The examples illustrate the invention:

### EXAMPLE 1

### Primary Screening for Compounds that Increase Pax4 Transcription

To perform high throughput screening for compounds that are either directly or indirectly capable of switching on Pax4 expression, a Pax4 reporter gene assay was established. Therefore, cDNA constructs containing a luciferase reporter gene under the control of the Pax4 promoter with the complete Pax4 gene locus were introduced into the human pancreatic duct cell line CAPAN-1.

Generation of stable Cell Lines and Drug Selections Capan-1 human pancreatic duct cell line ([HBT-79], purchased from American Type Culture Collection (ATCC), referred to as Capan-1 herein) were grown in high glucose DMEM [Gibco Cat. No. 61965-026] containing 10% FBS [Gibco Cat. No. 10270-106] at 37° C. under an atmosphere of 5% CO₂. The Pax-4-reporter gene construct (referred to as pKS-Pax-4-LUC)— a cDNA construct containing a luciferase reporter (LUC) gene under the control of the Pax4 promoter with the complete Pax4 gene locus were cloned into the plasmid pBlueskript ks (FIG. 5). The Minimal CMV reporter gene construct (referred to as pCMVmin-LUC): a fire fly luciferase reporter gene (LUC) under the control of a minimal CMV promotor (56 bp) was cloned into pcDNA3.1 plasmid (without promotor) (FIG. 6).

Two stable transfection of Capan-1 cells was performed by Lipofectamine 2000 (Invitrogen) method with 10% μg pPax-4-LUC and 2 μg pCMVmin-LUC. Cells (6*10⁶) were plated on 100-mm dishes in 8 ml growth media [DMEM high glucose+10% FBS] the night before transfection. The transfected cells were plated 24 h posttransfection by limiting dilution in media containing 500 μg/ml and 700 μg/ml Geneticin [Gibco Cat. No. 11811-098] and independent clones were isolated after 14 day's selection.

### Cellular HTS-Pax4 Screening Assay

Capan-1, stably transfected with pKS-Pax-4-LUC (clone18), cultivated without G418 for 4 day's, were plated at a density of 1×10⁴ cells per 96 well plate [Greiner, LIA plate white, Cat. No. 655083] in 100 μl DMEM containing 10% (v/v) FBS without G418 and grown for 24 hour. Cells were starved by serum starvation for 16 hour in 85 μl medium without FBS [DMEM (Cat. No. 25030-024) containing 5% L-glutamine]. Following serum starvation, 5 μl Trichostatin A (0.75 μM, [Sigma T8552; Lot. No. 111K4022],) and 10 μl of each LOPAC¹²⁸⁰ library compound (10 μM; LOPAC¹²⁸⁰, Library of Pharmacologically Active Compounds, Sigma. LO1280; Lot. No. 103K4703) were added to the cells and incubated at 37° C. for 30 hour. After 30 hours stimulation, 100 μl luciferase substrate britelite [PerkinElmer, Ultra-High Sensitivity, Cat. No. 6016976] was added and incubated for 2 min. The luminescence was measured within 15 minutes after reagent addition for maximum sensitivity.

### Counter Screen Assay

Stable transfected Capan-1-pCMVmin-LUC-54 cells, cultivated without G418 for 4 day's, were plated at a density of 4×10⁴ cells per 96 well plate [Greiner, LIA plate white, Cat. No. 655083] in 100 μl DMEM containing 10% (v/v) FBS and grown for 24 hour. Cells were starved by serum starvation for 16 hour in 8511 medium without FBS [DMEM(Cat. No. 25030-024) containing 5% L-glutamine]. Following serum starvation, the cells were dosed in triplicates by addition of 5 μl Trichostatin A (0.75 μM) and 10 μl of each selected compound (10 μM), dissolved in DMSO and incubated for 30 hour. After 30 hours stimulation, 100 μl luciferase substrate britelite [PerkinElmer, Ultra-High Sensitivity, Cat. No. 6016976] was added and incubated for 2 min. The luminescence was measured within 15 minutes after reagent addition for maximum sensitivity.

Analysis of mRNA by Reverse Transcription—PCR

After compound incubation under screening condition of Capan-1^{wt} and Capan-1-pKS-Pax-4-LUC-18 cells, mRNA was extracted by using the RNeasy Mini Kit (Qiagen). A Pax-4-Plasmid and genomic DNA was used as a control template. mRNA samples were pretreated with DNase to remove any traces of contamination of genomic DNA. First-stranded cDNA was synthesized by using Preamplification System for First Strand cDNA Synthesis kit (Gibco BRL). To confirm no contamination of genomic DNA, samples without reverse transcriptase treatment were prepared. Oligonucleotide primers used in this experiment: sense primer: TGC CTC TGG ATA CCC GGC AGC; antisense primer: CTC CM GAC ACC TGT GCG; (PCR-Product: 137 bp). The reactions were conducted in a DNA Thermal Cycler (Biometra) under the following conditions: Denaturation at 94° C. for 30 sec, annealing at 64° C. for 30 sec, and extension at 72° C. for 1 min. The number of cycles for Pax 4 was 40. PCR products were analyzed by agarose gel electrophoresis (3%) and ethidium bromide staining.

### Result

9-Bromo-7,12-dihydro-indolo[3,2-d][1]benzazepin-6(5H)-one (Kenpaullone) was identified as one of the compounds that were inducing the luciferase activity 6-fold. In the counterscreen, the induction found to be 1.2 fold.

### EXAMPLE 2

### Increase of Pax4 Transcription in Rat Insulinoma Cells

The response of the Pax4 gene to test compounds was investigated in the rat insulinoma cell line INS-1E. INS-1E cells are known to express Pax4 and to upregulate Pax4 levels in response to the treatment with activin-A and betacellulin. In the search for novel beta cell mitogens and/or beta cell protective agents the inventors treated INS-1E cells with different test compounds. Kenpaullone (concentration 20 μM) induces the relative Pax4 expression 8-fold compared to the control and compared to 1 nM activin A.

Further, human islets were treated with 5 μM Kenpaullone for 48 h. The treated islets show a 10-15 fold expression of Pax4 compared to untreated pancreatic islets. No effect was seen in small intestine and colon.

Alsterpaullone (20 μM) induces the relative Pax4 expression about 7-fold compared to the control. 1-Azakenpaullone (3 μM) induces the relative Pax4 expression about 6-fold. An induction of Pax4 expression was also found after treatment with GSK-3 inhibitors VIII (N-(4-Methoxybenzyl)-N′-(5-nitro-1,3-thiazol-2-yl)urea), ((2′Z,3′ E)-6-Bromoindirubin-3′-acetoxime), or (2′Z,3′E)-6-Bromoindirubin-3′-oxime).

### Quantitative RT-PCR

Total RNA from 8×10⁴ cells growing on 4 cm² surface area of a tissue culture dish was extracted using Qiagen RNAeasy kit according to the instructions of the manufacturer (Qiagen) and 2 μg was converted into cDNA. Primers for pax4, 18S RNA, and rat RNA polymerase II largest subunit (RPB1) were designed using the Primer Express 1.5 Software from Applied Biosystems and sequences can be obtained upon request. Quantitative real-time PCR was performed using Applied Biosystems SDS 7000 detection system. Amplifications from 2 independent experiments were performed in duplicate for each transcript and mean values were normalized to the mean value of the reference RNA 18S RNA.

### Cell Culture

INS-1E cells were cultured as described (Merglen, (2004) Endocrinology; 145: 667-678). Cells were seeded at a density of 2×10⁴ cells per cm² 6 to 8 days before the treatment with chemicals. During the growth period the medium was changed once. The cells were incubated for different periods of time with chemicals under serum-free conditions. The cells were harvested in Qiagen RNAeasy cell lysate buffer and immediately transferred to dry ice. The samples were stored at −20° C. until RNA isolation was carried out.

### EXAMPLE 3

### Increase of Pax4 Transcription in Rat Islets

The response of the Pax4 gene to two compounds was investigated in rat islets. Interestingly, 1-Azakenpaullone induces higher Pax4 RNA levels than activin-A, which serves as a technical control and has previously been shown to activate Pax4 transcription in rat islets. 1-Azakenpaullone transiently up-regulates Pax4 RNA levels, whereas treatment with or activin-A results in a more sustained increase in Pax4 RNA levels. No effects were observed on the expression levels of cyclophilin B and RNA polymerase II largest subunit (RPB1) indicating a specific regulation of the Pax4 gene.

### Quantitative RT-PCR

Total RNA from 200 to 400 islets growing in wells with 4 cm² surface area of a tissue culture dish was isolated according to instruction of the manufacturer of Trizol™ reagent. To further purify the RNA and to remove remaining DNA contaminations RNA was treated with the Qiagen RNAeasy kit according to the instructions of the manufacturer (Qiagen) and about 1 μg was converted into cDNA. Primers for pax4, 18S RNA, and rat RNA polymerase II largest subunit (RPB1) were designed using the Primer Express 1.5 Software from Applied Biosystems. Quantitative real-time PCR was performed using Applied Biosystems SDS 7000 detection system. Amplifications from at least 2 independent experiments were performed in duplicate for each transcript and mean values were normalized to the mean value of the reference RNA 18S RNA.

### Cell Culture

Rat islets were isolated from Wistar rats and cultured as described (Brun et al., 2004, JCB; 167: 1123-1135). In brief, 10 ml ice-cold liberase solution (Roche) was injected into the pancreas via the common bile duct. After dissection the pancreas was incubated for 40 minutes at 37° C. and then further dissociated by repeated pipetting using a 10 ml pipette. Islets were isolated by applying digested tissue to a ficoll-gradient before they were manually picked using a stereomicroscope. Islets were placed in bacteriological wells and compounds were administered as indicated. Islets were harvested in Trizol™ and immediately transferred to dry ice. The samples were stored at −20° C. until RNA isolation was carried out.

### EXAMPLE 4

### Protection of Beta Cells Against Apoptotic Signals

Beta cells are highly sophisticated cells capable of monitoring and balancing blood glucose levels. In type I diabetics most beta cells are destroyed by a T cell mediated autoimmune attack. In type II diabetics the situation is more complex and beta cell damage or loss are probably due to the synergistic effects of different factors stressing beta cells for long periods of time. For instance, chronic or recurrent exposure of beta cells to elevated serum levels of glucose and free fatty acids, which usually occurs in diabetics, are considered to cause beta cell dysfunction and loss.

Accordingly, in cell culture experiments beta cell apoptosis can be quickly induced by the combination of high concentrations of glucose and for example the fatty acid palmitate (glucolipotoxicity). Proinflammatory cytokines such as interleukin-1 beta (IL-1beta) and interferon-gamma (IFN-gamma) are also shown to harm beta cells by binding to their respective surface receptors present on the cell surface of beta cells. It turned out that insulinoma cells such as INS-1 cells are sensitive to apoptotic signals like primary beta cells and are therefore widely used to in the field to study glucolipotoxicity or cytokine induced beta cell death.

The beta cell line INS-1E was used to investigate anti-apototic effects of the compounds of the invention on beta cells. Apotosis was induced by culturing cells with a cytokine combination (interleukine-1beta/interferon gamma) or with high concentrations of both glucose and palmitate. 1-Azakenpaullone strongly (60%) inhibited cytokine-induced apoptosis (FIG. 7A), with a weaker effect (25%) against glucolipotoxicity induced apoptosis (FIG. 7B). The state of the treated and untreated cells was analyzed by microscopy. Cells that were exposed to toxic agents are clearly showing signs of cell death and 1-Azakenpaullone partially antagonizes this effect (FIG. 8).

### Cell Culture and Determination of DNA Fragmentation

INS-1E cells were cultured as described (Merglen et al., (2004) Endocrinology; 145: 667-678). Cells were seeded at a density of 1×10⁴ cells per 96-well in 96 well plates in 200 μl culture medium for 3 days. Then, cells were cultured for 1 day in 100 μl medium containing 1% FCS and 5 mM glucose. After an additional medium change cells were incubated for 1 hour in the presence of anti-apoptotic factors such as 1-Azakenpaullone. To induce apoptosis cells were then incubated with the cytokine combination IL-1 beta at 4 ng/ml and INF-gamma at 1 U/ml or the combination of glucose at 25 nM and palmitate at 0.04 nM for 24 hours. After removing the medium, the cells were washed with PBS and lysed in 50 μl lysis buffer. The rate of apoptosis was measured by the specific determination of mono- and oligonucleosomes in the cytoplasmic fraction of cell lysates using a cell death detection ELISA kit (ROCHE Cat. No. 1774425). The assay is based on a quantitative sandwich-enzyme-immunoassay-principle using mouse monoclonal anti-histone and anti-DNA peroxidase antibodies. The relative rate of apoptosis was photometrically determine by measuring the peroxidase activity of the immunocomplexes at 405 nm.

### EXAMPLE 5

### Promotion of Beta Cell Replication

The pancreatic beta cell mass is dynamic and is adjusted to the insulin need of the body. Accordingly, beta cell mass increases in pregnant females, in individuals that put on weight or in patients developing an insulin resistance. Beta cell mass is regulated by means of different mechanisms and an increase in beta cell mass can result from an increase in beta cell replication. Diabetes is associated with the loss of pancreatic beta cells and agents that are able to antagonize this process are of interest for the treatment of this disease.

Here, we demonstrate that the compounds of the invention stimulate the replication of beta cells in culture. We investigated the growth promoting effects by monitoring the percentage of replicating beta cells in isolated rat islets. Replicating beta cells were identified by immunohistological staining of dispersed islets with antibodies against C-peptide, a fragment of proinsulin, and the cell division marker Ki-67.

In this assay 1-Azakenpaullone and AR-AO14418 exhibit similar mitogenic activity on beta cells as the technical control, the peptide prolactin, a beta cell mitogen that drives beta cell expansion during pregnancy (FIG. 9A).

The combination of GSK3 inhibitors with other growth factors like nerve growth factor (NGF) or betacellulin can further boost the proliferative response of primary beta cells (FIG. 12). Furthermore, a number of structurally diverse and comparatively selective GSK3 inhibitors enhanced the rate of proliferating INS-1E cells as shown by BrdU incorporation and relative cell number counting (FIG. 13).

### In Vitro Beta Cell Proliferation Assay

Islets of Langerhans are isolated by standard Liberase digestion method from rat pancreata (Liberase™ Cl enzyme blend BMB Cat. # 1814-435, ROCHE).

Freshly isolated islets are cultured in vitro with or without the addition of the factor of interest for 48 h. Following the culture period the islets are dispersed gently by titration in Ca²⁺ and Mg²⁺ free PBS. The resulting single cell suspension is applied to adhesive slides at 3000-6000 cells per well (Adhesion slides/Fa Superior Marienfeld REF 09 000 00/). The adherent islet cells are fixed and stained by standard immunofluorescence techniques for C-peptide, a fragment of proinsulin and Ki-67 a marker of proliferating cells.

An Olympus microscope equipped with an automatic image acquisition device (Olympus) is used for counting of C-peptide positive beta cells. Proliferating C-peptide/Ki-67 double positive beta cells are counted manually. Thereby the fraction of proliferating beta cells can be determined.

The structurally diverse GSK3 inhibitor CHIR99021 also promotes replication of primary rat beta cells (FIG. 14). Interestingly, the combination of GSK3 inhibitors with the incretin GIP (gastrin inhibitory protein)) further increased the proliferative response of primary rat beta cells (FIG. 14).

The fact that structurally diverse GSK3 inhibitors promote beta cell replication of INS-1E cells as well as of primary rat beta cells evidence that GSK3 is the critical target regulating growth of beta cells. To confirm this assumption GSk3 α and β expression was suppressed in INS-1E cells through the transfection of gene specific siRNA duplexes (FIG. 15). FIG. 15B illustrates that only INS-1E cells expressing reduced levels of both GSK3 isoforms proliferate at a higher rate than INS-1E cells expressing wild type levels of GSK3.

### RNA Interference, Western Blotting and Proliferation Assay

For western blotting INS-1E cells were seeded in 12 well plates at a density of 2×10⁵ cells per well and cultured o/n before transfection. Cells were transfected with 6 μl of HiPerFect Transfection Reagent (Qiagen) mixed with 5 nM siRNA duplexes (Qiagen). For quantitative PCR cells were harvested in Qiagen lysis buffer after 48 hours. Four or six days after transfection, cells were harvested in a standard protein lysis buffer for immunoblotting. For western blotting, depending on the size of the gel 2 to 20 μg of protein was loaded per lane, separated by SDS-Page and immunoblotted by standard methods well described in the art. The following antibodies were used: mouse monoclonal anti-GSK3 α and β (Calbiochem Cat. Nr. 368662) at a 1:1000 dilution, mouse monoclonal anti-γ-tubulin (Sigma Cat. Nr. T6557) at a 1:5000 dilution and as a secondary antibody HRP-conjugated goat anti-mouse antibody (Pierce Prod. Nr. 34075) at a 1:10000 dilution. The immunoblot was developed using the chemiluminescence detection system SuperSignal West Dura from Pierce (Prod. Nr. 34075).

For the proliferation assay INS-1E cells were seeded in 96 well plates at a density of 2×10⁴ cells per well and cultured o/n before transfection. Cells were transfected with 0.35 to 0.7 μl of HiPerFect Transfection Reagent (Qiagen) mixed with 5 nM siRNA duplexes. After 48 hours incubation medium was replaced by starvation medium which contains 1% FCS instead of 5%. 24 hours later BrdU labeling solution (Roche) was added to the medium for 4 hours and the rate of proliferating cells was then determined using the Cell Proliferation ELISA assay (Roche) according to the instructions of the manufacturer. Chemiluminescence was measured using the Analyst™ HT detection system from LJL Biosystems Inc. The following siRNA duplexes were purchased from Qiagen:

rat GSK3 β sense r(CGAUUACACGUCUAGUAUA)dTdT,
antisense r(UAUACUAGACGUGUAAUCG)dGdT;
rat GSK3 α sense r(GGGUGUAAAUAGAUUGUUA)dTdT,
antisense r(UAACAAUCUAUUUACACCC)dAdA; control non-silencing siRNA sense UUCUCCGAACGUGUCACGUdTdT, antisense ACGUGACACGUUCGGAGAAdTdT. As a second independent siRNA control luciferase GL3 siRNA was used, sense CUUACGCUGAGUACUUCGAdTdT, antisense UCGAAGUACUCAGCGUAAGdTdT.

### EXAMPLE 6

### Generation of Stable Cell Lines

INS-1E cell lines were grown as described below. The Pax-4-reporter gene construct (referred to as pKS-Pax-4-LUC), a cDNA construct containing a firefly luciferase reporter (LUC) gene under the transcriptional control of human Pax4 promoter sequences and the nearly complete human Pax4 gene locus (cf. FIG. 5A) was cloned into the plasmid pBluescript ks.

Adherent INS-1E cells were transfected using Lipofectamine 2000 (Invitrogen) according to the protocol provided by the supplier. The transfected cells were treated with media containing 400 μg/ml G418 Sulfate (e.g. Geneticin from Gibco; Cat. No. 11811-098), a selection agent for eukaryotic cells, 24 to 48 hours after the transfection. To establish independent stable cell lines, cell colonies which occurred 2 to 6 weeks after the transfection were individually transferred to new culture dishes and then propagated like untransfected INS-1E cells, but in the presence of G418 Sulfate.

### EXAMPLE 7

### Activin B Increases Pax4 Transcription

The response of the Pax4 gene to mitogens was investigated in the rat insulinoma cell line INS-1E. INS-1E cells are known to express Pax4 and to upregulate Pax4 levels in response to the treatment with activin-A and betacellulin (Ueda (2000), supra, Li et al. (2004) supra, Brun et al. (2004), supra). In the search for novel beta-cell mitogens the inventors treated INS-1E cells with proteins related to activin A or betacellulin. Activin B an activin AB were found to be almost equally potent in stimulating Pax4 transcription as activin A. Other TGF-beta family members, such as BMP 4 and 7 which are known to recognize the activin-receptor type II subunit of the heterodimeric activin receptor hardly induced Pax4 gene transcription. Maximal Pax4 induction was observed with 1 nM activin B that induced about a 7.5-fold increase in Pax4 levels. The Pax4 RNA expression level was normalized to this of 18S RNA. The level of the unrelated gene RNA polymerase II largest subunit (RPB1) was unaffected by activin B treatment.

FIG. 16 illustrates a representative experiment in which the relative Pax4 levels were quantified using quantitative real time RT-PCR. As shown in FIG. 16, activin B and activin AB induce Pax4 gene transcription in INS-1E cells. Quantitative real-time RT-PCR was done with RNA isolated from INS-1E cells cultured under conditions as described below. Low levels of Pax4 are expressed in INS-1E cells. Data are presented as relative levels to the basal Pax4 expression level in untreated INS-1E cells (Co.). The values for untreated INS-1E (Co.) and activin-B are averages of three experiments enabling the determination of standard deviations; the other values are averages of two experiments.

### Quantitative RT-PCR

Total RNA from 8×10⁴ cells growing on 4 cm² surface area of a tissue culture dish was extracted using Qiagen RNAeasy kit according to the instructions of the manufacturer (Qiagen) and 2 μg was converted into cDNA. Primers for pax4, 18S RNA, and rat RNA polymerase II largest subunit (RPB1) were designed using the Primer Express 1.5 Software from Applied Biosystems and sequences can be obtained upon request. Quantitative real-time PCR was performed using Applied Biosystems SDS 7000 detection system. Amplifications from 2 independent experiments were performed in duplicate for each transcript and mean values were normalized to the mean value of the reference RNA 18S RNA.

### Cell Culture

INS-1E cells were cultured as described (Merglen, (2004) Endocrinology; 145: 667-678). Cells were seeded at a density of 2×10⁴ cells per cm² 6 to 8 days before the treatment with chemicals. During the growth period the medium was changed once. The cells were incubated for different periods of time with chemicals under serum-free conditions. The cells were harvested in Qiagen RNA easy cell lysate buffer and immediately transferred to dry ice. The samples were stored at −20 degree until RNA isolation was carried out.

### EXAMPLE 8

### Identification and/or Characterization of Beta-Cell Mitogens

For the identification and/or characterization of beta-cell mitogens a Pax4 reporter gene assay has been established. The rat insulinoma cell line INS-1E was stably transfected with a DNA construct containing the luciferase reporter gene under the transcriptional control of the human Pax4 promoter and the complete Pax4 gene locus (FIG. 5).

Luciferase activity that can be quantified using appropriate imaging systems reflects the activation status of the human Pax4 promoter. Two types of cell lines reacting either to activins or small molecule kinase inhibitors have been identified so far.

INS-1-cl-1.5 cells show Pax4 reporter gene activity upon treatment with small molecule kinase inhibitors as exemplified in FIG. 17. Maximal Pax4 reporter gene activity observed was about 3-fold above the level of untreated cells (Contr.) after 48 hour treatment with inhibitor 11. Similar levels of activation were observed after 24 and 62 hours of incubation with inhibitor II (data not shown). The assay signal dynamic range is between 2 and 3. This clone, however, does not react to Activin stimulation indicating the existence of at least two independent signalling pathways regulating Pax4 transcription.

The second type of clones identified react to Activin treatment but not to incubation with the kinase inhibitors 11 (FIG. 19), II and III (data not shown). Two independent clones named INS-1-cl.-3.5 or INS-1-cl.-9 showed this pattern of Pax4 regulation. The reason for the different regulation of the human Pax4 promoter observed in the different cell lines is unknown. Here, representative data generated with the cell line INS-1-cl.-3.5 are presented. Most importantly, the treatment of the reporter cell line with the Activin related protein TGF-beta 1 does not effect Pax4 transcription (FIG. 18). The assay signal dynamic range is somewhere between 0.5 and 1 (FIG. 18).

1. Use of a Pax4 stimulating compound and optionally an immunosuppressive agent for the manufacture of a pharmaceutical composition for the prevention and/or treatment of pancreatic autoimmune disorders.

2. The use of claim 1 for the prevention and/or treatment of autoimmune diabetes.

3. Use of a Pax4 stimulating compound and optionally an immunosuppressive agent for the manufacture of a pharmaceutical composition for the prevention and/or treatment of type I diabetes, LADA (latent autoimmune diabetes in adults), or late stages of diabetes type II.

4. Use of a Pax4 stimulating compound and optionally an immunosuppressive agent for the manufacture of a pharmaceutical composition for promoting the growth and/or survival of pancreatic beta cells.

5. Use of a GSK3-inhibitor and optionally an immunosuppressive agent for the manufacture of a pharmaceutical composition for promoting the expression of the transcription factor Pax4 in pancreatic beta cells.

6. The use of claim 1 wherein the compound is a paullone.

7. The use of claim 6 wherein the compound is a compound of formula (I): 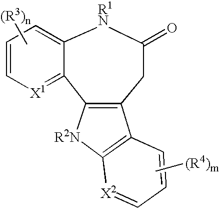
wherein X1 and X2 are independently N or CR3 and preferably X1 is N or CH and X2 is CH;
R1 and R2 are independently H, —C₁-C₆ alkyl, optionally substituted, or —CO—C₁-C₆ alkyl, optionally substituted, wherein the substituents are independently selected from one or more of halo, CN, OH, O—C₁-C₆ alkyl; COOH, COO—C₁-C₆ alkyl, —CONH₂, —CONH(C₁-C₆)alkyl, —CON(C₁-C₆ alkyl)₂, aryl, heteroaryl or combinations thereof;
each R3 and R4 is independently selected from C₁-C₆ alkyl, —C₂-C₆ alkenyl; —C₂-C₆ alkynyl; —C₃-C₁₀ cycloalkyl, —C₃-C₁₀ heterocyclyl,
aryl with 6 to 10 carbon atoms, heteroaryl with 5 to 10 ring atoms; each optionally substituted; halo, e.g. F, Cl, Br or I; —NO₂, —CN, —OR1; —COOR1 or —NR1R2;
wherein R1 and R2 are as defined above; and
wherein alkyl, alkenyl or alkynyl is optionally substituted with one or more of halo, —NO₂, —CN, —OR1, COOR1, —OCOR1, —NR1R2, NR1COR2, —NR1OCOR2, —NR1CONR1R2, —SR1, SOR1, —SO₂R1, —SONR1R2, SO₂NR1R2 or —NR1SO₂NR1NR2; or combinations thereof, wherein R1 and R2 are as defined above;
wherein cycloalkyl, heterocyclyl, aryl or heteroaryl is optionally substituted with one or more of C₁-C₆ alkyl, halo, —NO₂, —CN, —OR1, COOR1, —OCOR1, —NR1R2, NR1COR2, —NR10COR2, —NR1CONR1R2, —SR1, SOR1, —SO₂R1, —SONR1R2, SO₂NR1R2 or —NR1SO₂NR1NR2; or combinations thereof, wherein R1 and R2 are as defined above;
or wherein two R3 or two R4 may together form a ring;
n=0-3, preferably 0-1 and more preferably 0;
m=0-3, preferably 0, 1 or 2 and more preferably 1 or 2;
or an optical isomer or a salt thereof.

8. The use of claim 1 wherein the compound is an indirubin.

9. The use of claim 8 wherein the compound is a compound of formula (II): 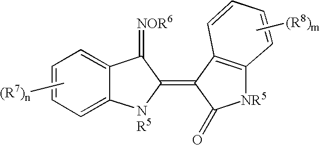
wherein R5 and R6 are independently H, —C₁-C₆ alkyl, optionally substituted, or —CO—C₁-C₆ alkyl, optionally substituted, wherein the substituents are independently selected from one or more of halo, CN, OH, O—C₁-C₆ alkyl; COOH, COOC₁-C₆ alkyl, —CONH₂, —CONH(C₁-C₆ alkyl), —CON(C₁-C₆ alkyl)₂, aryl, heteroaryl or combinations thereof;
each R7 and R8 is independently selected from C₁-C₆ alkyl, —C₂-C₆ alkenyl; C₂-C₆ alkynyl; C₃-C₁₀ cycloalkyl, —C₃-C₁₀ heterocyclyl, aryl with 6 to 10 carbon atoms, heteroaryl with 5 to 10 ring atoms, each optionally substituted; halo, e.g. F, Cl, Br or I; —NO₂, —CN, —OR1; —COOR1; or NR1R2, wherein R1 and R2 are as defined in formula (I),
wherein alkyl, alkenyl or alkynyl is optionally substituted with one or more of oxo, halo, —NO₂. —CN, —OR1, COOR1, —OCOR1, —NR1R2, NR1COR2, —NR1OCOR2, —NR1CONR1R2, —SR1, SOR1, —SO₂R1, —SONR1R2, SO₂NR1R2 or —NR1SO₂NR1NR2 or combinations thereof, wherein R1 and R2 are as defined in formula (I);
wherein cycloalkyl, heterocyclyl, aryl or heteroaryl is optionally substituted with one or more of C₁-C₆ alkyl, oxo, halo, —NO₂. —CN, —OR1, COOR1, —OCOR1, —NR1R2, NR1COR2, —NR1OCOR2, —NR1CONR1R2, —SR1, SOR1, —SO₂R1, —SONR1R2, SO₂NR1R2 or —NR1SO₂NR1NR2 or combinations thereof, wherein R1 and R2 are as defined in formula (I);
or two R7 or two R8 may together form a ring;
n=0-3, preferably 0-1 and more preferably 0;
m=0-3, preferably 0-1 and more preferably 1,
or an optical isomer or a salt thereof.

10. The use of claim 1 wherein the compound is a substituted urea.

11. The use of claim 10 wherein the compound is a compound of formula (III): 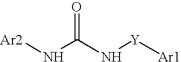
wherein Y is —[C(R9)₂]ᵣ, each R9 is independently H, F or CH₃, and r is 0-3, and Ar1 and Ar2 are aromatic or heteroaromatic rings optionally substituted with at least one R7 wherein each R7 is independently selected from C₁-C₅ alkyl, optionally halogenated; halo, e.g. F, Cl, Br or I; —NO₂, —CN, —OR5; —COOR5; —OCOR5; —NR5R6 and —NR5COR6 and wherein R5 and R6 are independently H, C₁-C₅ alkyl, optionally halogenated, or —CO—C₁-C₅ alkyl.

12. The use of claim 1, wherein the compound is an ethylene diamino derivative.

13. The use of claim 12, wherein the compound is a compound of formula (IV): 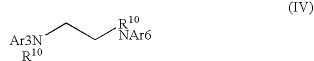
wherein each R10 is independently H, C₁-C₆ alkyl optionally substituted or —CO—C₁-C₆ alkyl optionally substituted, wherein the substituents are as defined for the substituents of R1 and R in formula (I),
Ar3 is an aromatic or heteroaromatic ring, preferably a 6-membered aromatic or heteroaromatic ring, more preferably a pyridine ring, e.g. a −2-pyridyl radical optionally substituted at least once, preferably once or twice, with R7 as defined for formula (II), wherein R7 is preferably selected from —NO₂, —NR5R6, CN and combinations thereof, wherein R5 and R6 are as defined in formula (II) and wherein R5 and R6 are preferably H,
Ar4 is an aromatic or heteroaromatic ring, preferably a 6-membered aromatic or heteroaromatic ring, more preferably a pyridine or a pyrimidine ring, e.g. a 2-pyridyl or a 2-pyrimidinyl radical substituted at least once, preferably once or twice with a cyclic radical selected from aryl with 6-10 carbon atoms, heteroaryl with 5-10 carbon atoms, cycloalkyl with 3-10 carbon atoms and heterocyclyl with 3-10 ring atoms, wherein the aromatic heteroaromatic ring and/or its cyclic substituents are optionally substituted at least once with R7 as defined in formula (II), wherein R7 is preferably selected from H, C₁-C₅ optionally halogenated and oxo, or an optical isomer or salt thereof.

14. The use of claim 1 wherein the compound is a maleimide derivative.

15. The use of claim 14, wherein the compound is a compound of formula (VII): 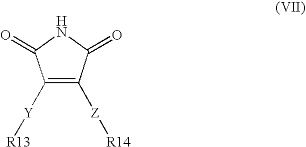
wherein R13 and R14 are independently selected from C₁-C₆ alkyl, —C₂-C₆ alkenyl, —C₂-C₆ alkynyl, —C₃-C₁₀ cycloalkyl, —C₃-C₁₀ heterocyclyl, aryl with 6 to 10 carbon atoms and heteroaryl with 5 to 10 ring atoms, each optionally substituted, wherein alkyl, alkenyl or alkynyl is optionally substituted with one or more of oxo, halo, —NO₂, —CN, —OR1, COOR1, —OCOR1, —NR1R2, NR1COR2, —NR10COR2, —NR1CONR1R2, —SR1, SOR1, —SO₂R1, —SONR1R2, SO₂NR1R2 or —NR1SO₂NR1NR2 or combinations thereof, wherein R1 and R2 are as defined in formula (I);
wherein cycloalkyl, heterocyclyl, aryl or heteroaryl is optionally substituted with one or more of C₁-C₆ alkyl, oxo, halo, —NO₂, —CN, —OR1, COOR1, —OCOR1, —NR1R2, NR1COR2, —NR1OCOR2, —NR1CONR1R2, —SR1, SOR1, —SO₂R1, —SONR1R2, SO₂NR1R2 or —NR1SO₂NR1NR2 or combinations thereof, wherein R1 and R2 are as defined in formula (I);
X and Y are independently selected from a chemical bond, NR1, O and S, wherein R1 is as defined in formula (I) or an optical isomer or a salt thereof.

16. The use of claim 1 wherein the immunosuppressive agent is selected from the compounds as shown in Table 1 or combinations thereof, particularly from DiaPep277, anti-CD-3-antibodies and/or GAD peptides.

17. The use of claim 1 in combination with at least one further beta cell mitogen and/or beta cell protective agent, particularly GLP-1 or derivatives thereof, exendin, prolactin, neurotrophins or combinations thereof.

18. The use of claim 1 for the protection, survival and/or regeneration of insulin producing cells, particularly insulin producing beta cells.

19. The use of claim 1 for the promotion of the differentiation of insulin producing cells, particularly insulin producing beta cells.

20. The use of claim 19 for the generation of beta cells from stem cells, particularly from embryonic stem cells.

21. The use of claim 19 for the generation of beta cells from duct cells or exocrine pancreatic cells.

22. The use of claim 1 wherein the pharmaceutical composition is for administration to a patient in need thereof.

23. The use of claim 22 wherein the pharmaceutical composition is for administration to a patient who is to receive or has received transplantation of pancreatic tissue.

24. The use of claim 1 wherein the pharmaceutical composition is for administration to beta cells or progenitor cells thereof ex vivo.

25. The use of claim 24 for generating replacement material for dysfunctional and/or destroyed beta cells.

26. The use of claim 24 for the manufacture of a transplantable beta cell preparation.

27. A method of identifying and/or characterizing a pancreatic beta-cell mitogen comprising the steps:
(i) providing a cell which is transfected with a reporter gene construct comprising a reporter gene which is operatively linked to an expression control sequence of a pancreatic gene or a gene whose function is controlled by a pancreatic gene, preferably the Pax4 gene,
(ii) contacting said cell with a compound and
(iii) determining reporter gene expression in said cell as a response to the presence of the compound.

28. The method of claim 27, wherein said cell is capable of regulating the expression level of a pancreatic gene or a gene whose function is controlled by a pancreatic gene and is preferably of pancreatic origin, derived from a beta cell or a precursor cell thereof.

29. The method of claim 27, wherein said cell is an insulinoma cell, preferably of mammalian origin.

30. The method of claim 27, wherein said cell is a cell which is responsive to treatment with kinase inhibitors and/or to treatment with activin A, B, AB, C, D, TGF-beta, HGF, IGF, prolactin, GLP-1 or derivatives thereof, EGF, betacellulin, glucose.

31. The method of claim 27, wherein said expression control sequence is of mammalian, preferably human, origin.

32. The method of claim 27, wherein said reporter gene encodes a gene product which can be determined by optical or enzymatic methods.

33. The method of claim 27, wherein said reporter gene comprises a sequence coding for firefly luciferase, chloramphenicol acetyltransferase or beta galactosidase.

34. The method of claim 27, wherein said insulin producing cell is stably transfected with the reporter gene construct.

35. The method of claim 27, wherein a plurality of compounds is tested in parallel.

36. The method of claim 27, wherein at least one step is carried out automatically.

37. The method of claim 28, further comprising preparing a pharmaceutical preparation which comprises as an active ingredient a pancreatic beta-cell mitogen identified and/or characterized according to steps (i)-(iii) or a compound derived therefrom.

38. Insulinoma cell which is transfected with a reporter gene construct comprising a reporter gene which is operatively linked to an expression control sequence of a pancreatic gene or a gene whose function is controlled by a pancreatic gene, preferably the Pax4 gene, preferably comprising a Pax4 promoter and at least part of the Pax4 gene locus.

39. The cell of claim 38, wherein said expression control sequence is of mammalian, preferably human origin.

40. The cell of claim 38, wherein said insulinoma cell is of mammalian origin, preferably of rat, mouse or human origin.

41. The cell of claim 38 which is responsive to treatment with kinase inhibitors and/or to treatment with activin A.

42. The cell of claim 38, which is stably transfected.

43. The cell of claim 38, wherein said reporter gene comprises a sequence coding for firefly luciferase, chloramphenicol acetyltransferase or beta galactosidase.

44. Test system comprising an insulinoma cell which is stably transfected with a reporter gene construct comprising a reporter gene operatively linked to an expression control sequence of a pancreatic gene or a gene whose function is controlled by a pancreatic gene preferably the Pax4 gene and at least one positive or negative control compound.

45. The test system of claim 44, wherein said expression control sequence is of mammalian, preferably human, origin.

46. The test system of claim 44, wherein said reporter gene comprises a sequence coding for firefly luciferase, chloramphenicol acetyltransferase or beta galactosidase.

47. The test system of claim 44, wherein said positive or negative control compounds are selected from activins A, B, AB, C, D, TGF-beta, HGF, IGF, prolactin, GLP-1 or derivatives thereof, EGF, betacellulin, glucose, small molecule kinase inhibitor, inhibitor I, II, III or a combination thereof.

48. A method of preventing and/or treating a pancreatic autoimmune disorder in a patient in need thereof, the method comprising administering to the patient an effective amount of a Pax4 stimulating compound.
